(19) United States
(12) Patent Application Publication
(10) Pub. No.: US 2023/0000858 A1
(43) Pub. Date: Jan. 5, 2023

(54) Nanoliposomal Irinotecan for Use in Treating Small Cell Lung Cancer

(71) Applicant: Ipsen Biopharm Ltd., Wrexham (GB)

(72) Inventors: Bambang Adiwijaya, Belmont, MA (US); Jonathan Basil Fitzgerald, Arlington, MA (US); Helen Lee, Cambridge, MA (US)

(73) Assignee: Ipsen Biopharm Ltd., Wrexham (GB)

(21) Appl. No.: 17/699,374

(22) Filed: Mar. 21, 2022

Related U.S. Application Data
(63) Continuation of application No. 16/302,050, filed on Nov. 15, 2018, now Pat. No. 11,318,131.
(60) Provisional application No. 62/474,661, filed on Mar. 22, 2017.
(60) Provisional application No. 62/455,823, filed on Feb. 7, 2017.
(60) Provisional application No. 62/433,925, filed on Dec. 14, 2016.
(60) Provisional application No. 62/422,807, filed on Nov. 16, 2016.
(60) Provisional application No. 62/415,821, filed on Nov. 1, 2016.
(60) Provisional application No. 62/414,050, filed on Oct. 28, 2016.
(60) Provisional application No. 62/394,870, filed on Sep. 15, 2016.
(60) Provisional application No. 62/370,449, filed on Aug. 3, 2016.
(60) Provisional application No. 62/362,735, filed on Jul. 15, 2016.
(60) Provisional application No. 62/345,178, filed on Jun. 3, 2016.
(60) Provisional application No. 62/337,961, filed on May 18, 2016.

(51) Int. Cl. A61K 31/4745; A61P 35/00; A61K 9/00; A61K 9/127; A61K 31/573

(52) U.S. Cl. CPC A61K 31/4745; A61P 35/00; A61K 9/0019; A61K 9/127; A61K 31/573; A61K 33/243

(57) ABSTRACT

Novel therapies for the treatment of small cell lung cancer (SCLC) include the administration of an antineoplastic therapy consisting of liposomal irinotecan administered once every two weeks, optionally including the administration of other non-antineoplastic agents to the patient such as the administration of a corticosteroid and an anti-emetic to the patient prior to the administration of the irinotecan liposome.

### CROSS-REFERENCE TO RELATED APPLICATIONS

This application is a continuation application of U.S. patent application Ser. No. 16/302,050 (filed Nov. 15, 2018), which is a national phase entry pursuant to 35 U.S.C. § 371 of International Application No. PCT/IB2017/000681 (filed May 17, 2017), which claims benefit of U.S. Provisional Application No. 62/337,961 (filed May 18, 2016), U.S. Provisional Application No. 62/345,178 (filed Jun. 3, 2016), U.S. Provisional Application No. 62/362,735 (filed Jul. 15, 2016), U.S. Provisional Application No. 62/370,449 (filed Aug. 3, 2016), U.S. Provisional Application No. 62/394,870 (filed Sep. 15, 2016), U.S. Provisional Application No. 62/414,050 (filed Oct. 28, 2016), U.S. Provisional Application No. 62/415,821 (filed Nov. 1, 2016), U.S. Provisional Application No. 62/422,807 (filed Nov. 16, 2016), U.S. Provisional Application No. 62/433,925 (filed Dec. 14, 2016), U.S. Provisional Application No. 62/455,823 (filed Feb. 7, 2017), and U.S. Provisional Application No. 62/474,661 (filed Mar. 22, 2017), each of which is incorporated herein by reference in its entirety.

### TECHNICAL FIELD

The present invention relates to the treatment of patients diagnosed with small cell lung cancer (SCLC), including patients with SCLC disease progression after treatment with a platinum-based therapy.

### BACKGROUND

Small-cell lung cancer (SCLC) is a highly malignant cancer that most commonly arises within the lung, although it can arise in other body sites. SCLC usually presents as large, rapidly developing lesions arising from the centrally located tracheobronchial airways and invading the mediastinum. Typically, patients present with a cough or dyspnea, wheezing, and/or chest pain. Weight loss, fatigue and anorexia occur in up to one third of the patients. At the time of diagnosis, two thirds of the patients with SCLC have one or more clinically detectable distant metastases.

Initial (first line) treatment of SCLC can include administration of a platinum-based therapy such as 4-6 treatment cycles of cisplatin or carboplatin, in combination with etoposide or irinotecan. Current subsequent (second line) therapy upon SCLC disease progression (after first line therapy) has been reported to provide overall survival of about 7.7 months (sensitive patients) and 5.4 months (refractory patients) based on (Owonikoko, T K, et al., J Thorac Oncol. 2012 May; 7(5):866-72). One second line therapy is the administration of topotecan (e.g., HYCAMTIN, topotecan hydrochloride injection), reported in certain regimens to provide an overall survival of 7.8 months (9.9 months in sensitive patients, 5.7 months in refractory patients) (Owonikoko, T K, et al., J Thorac Oncol. 2012 May; 7(5):866-72). For example, of second line SCLC treatment with topotecan at 1.5 mg/m² administered on days 1-5 once in a three (3)-week treatment cycle provided overall response rates of about 7-24%, progression free survival (PFS) of about 3.1-3.7 months, and overall survival (OS) of 5.0-8.9 months (accompanied by grade 3 or greater neutropenia rates of 28-88% and grade 3 or greater diarrhea of less than about 5%) (PMIDs 16481389, 17135646, 17513814, 9164222, 10080612, 25385727). Another reported SCLC second line therapy is the administration of non-liposomal irinotecan at 300 mg/m² once every three (3) weeks, providing mixed overall response rates of 0-33%, PFS of 1.7-2.8 months, and OS of 4.6-6.9 months (accompanied by grade 3 or greater neutropenia rates of 21-23% and grade 3 or greater diarrhea of less than about 0-13%) (PMID 19100647, 1321891).

Irinotecan is an active agent in the treatment of SCLC (e.g., listed in NCCN and ESMO guidelines) but it is not approved in the US or EU. Furthermore, it failed in a PHASE III registration-directed study in combination with a platinum in first line SCLC (PMID: 16648503). No targeted treatment has been successful to date in significantly improving the outcome of patients. The research of novel treatments for this disease is therefore urgently needed.

### SUMMARY

The present disclosure provides methods of treating patients with small cell lung cancer after disease progression following platinum-based therapy, by administering a therapeutically effective amount of liposomal irinotecan. In particular, a liposomal irinotecan such as MM-398 (ONIVYDE), can be administered once every two weeks to patients diagnosed with SCLC after disease progression following platinum-based therapy. In some embodiments, the liposomal irinotecan can be administered to patients diagnosed with SCLC disease progression on or after first-line platinum based chemotherapy (carboplatin or cisplatin), immunotherapy, and/or chemo-radiation including platinum-based chemotherapy for treatment of limited or extensive stage SCLC.

A human patient diagnosed with small cell lung cancer (SCLC) after disease progression following platinum-based therapy for the SCLC can be treated once every two weeks with an antineoplastic therapy consisting of a single dose of 90 mg/m² of irinotecan (free base) encapsulated in irinotecan liposomes. In another embodiment, a human patient who is known to be homozygous for the UGT1A1*28 allele and is diagnosed with small cell lung cancer (SCLC) after disease progression following platinum-based therapy for the SCLC can be treated with an antineoplastic therapy consisting of a single reduced dose (e.g., 50-70 mg/m², including 50 mg/m² or 70 mg/m²) of irinotecan (free base) encapsulated in liposomes, administered once every two weeks. In another embodiment, a human patient who has previously experienced a Grade 3+ adverse event while or after receiving liposomal irinotecan after being diagnosed with small cell lung cancer (SCLC), and after disease progression following platinum-based therapy for the SCLC, can be treated with an antineoplastic therapy consisting of a single reduced dose (e.g., 50-70 mg/m², including 50 mg/m² or 70 mg/m²) of irinotecan (free base) encapsulated in liposomes, administered once every two weeks.

The liposomal irinotecan can be a pharmaceutically acceptable liposome formulation of irinotecan, comprising irinotecan in a delivery form having a diameter of about 100 nm, such as a liposomal irinotecan (Example 1). Various suitable liposomal irinotecan preparations can be manufactured as disclosed herein (Example 8). Preferably, the liposomal irinotecan is the product MM-398 (ONIVYDE®) (Example 9). In the present disclosure MM-398 is used interchangeably with MM-398 liposomal irinotecan.

### BRIEF DESCRIPTION OF THE DRAWINGS

FIG. 1 is a graph showing the drug sensitivity data to SN-38 from the Sanger database were plotted for SCLC, gastrointestinal, and pancreatic cancer cell lines (Example 2).

FIGS. 2A and 2B are kinetic growth curves of DMS114 and NCI-H1048 SCLC cell lines acquired on an Incucyte instrument over 88 hours at various SN-38 concentrations

FIG. 3 is a graph showing the anti-tumor activity of MM-398 in the DMS114 xenograft model of SCLC. MM-398 was administered IV at 10 or 20 mg/kg irinotecan hydrochloride trihydrate starting on Day 23 and given weekly for 4 weeks and compared to saline control (black circles).

FIG. 4 is a Kaplan-Meier Plot of overall survival by quartiles of unencapsulated SN-38 (uSN38) time above threshold in the MM-398+5FU/LV arm of NAPOLI-1. Q1-Q4 represent the quartiles of uSN38 time above threshold. Q1 represents the shortest time and Q4 represents the longest time.

FIG. 5 is a graph showing the association between best response and duration of uSN38>0.03 ng/mL for MM-398+5FU/LV arm in NAPOLI-1.

FIG. 6A is a graph showing the association between unencapsulated SN-38 Cmax and Neutropenia Grade≥3 in patients treated with MM-398.

FIG. 6B is a graph showing the association between total irinotecan Cmax and Diarrhea grade ≥3 in patients treated with MM-398.

FIG. 7A is a graph showing carboxylesterase (CES) activity; increased tumor SN-38 levels were associated with increased tumor deposition, as assessed by tumor CPT-11 at 24 h post administration in SCLC mouse xenograft models.

FIG. 7B is a graph showing carboxylesterase (CES) activity; SCLC PDX tumors have CES activity comparable to other indications in which irinotecan is active.

FIG. 7C is a graph showing cell sensitivity; Nal-IRI tumor deposition is consistent with range of SN-38 sensitivity in H1048 SCLC cells.

FIG. 7D is a graph showing cell sensitivity; cytotoxicity of Topo1 inhibitors increases with exposure.

FIG. 7E is a chart showing that topotecan administration is severely limited by toxicity, thus limiting sustained inhibition of topo1 in comparison to Onivyde mediated prolonged SN-38 exposure.

FIG. 8A shows the anti-tumor activity of MM-398 in the DMS-53 xenograft model of SCLC.

FIG. 8B shows the anti-tumor activity of MM-398 in the HCl-H1048 xenograft model of SCLC.

FIG. 8C shows the percent survival of rats in the H841 rat orthotopic xenograft model of SCLC that are treated with control, Onivyde (30 or 50 mg/kg salt), irinotecan (25 mg/kg) or topotecan (4 mg/kg) at days post inoculation.

FIGS. 9A and 9B are graphs showing the tumor metabolite levels in SCLC xenograft models treated with MM-398 and nonliposomal Irinotecan. At 24 hours post-injection, (FIG. 9A) CPT-11 and (FIG. 9B) active metabolite SN-38 in tumors were significantly higher for mice treated with MM-398 at 16 mg/kg (salt) compared to nonliposomal irinotecan at 30 mg/kg (salt).

FIGS. 10A and 10B are graphs showing that Nal-IRI is superior to all comparator treatment arms in treatment naïve SCLC xenograft models: FIG. 10A is a graph showing Treatment Naïve SCLC Model NCl-H1048 (Nal-IRI 16 mg/kg clinically equivalent dose per BSA=1×˜90 mg/m² MM-398; Topotecan 0.83 mg/kg/wk, D1-2, q2w clinically equivalent dose per BSA=1×˜1.5 mg/m² Topotecan, q3w, D1-5); FIG. 10B shows the number of complete response (Nal-IRI). NCI-H1048 is a chemo-sensitive model (established from pleural effusion metastases of SCLC). All nal-IRI-treated animals have complete response (CR) after 2-3 doses—but dose response is observed at early time-point. IRI-treated animals progress after initially responding to treatment; while nal-IRI treated animals remain CR to date.

FIGS. 11A and 11B describe a 2L SCLC xenograft model created through treatment with Carboplatin+Etoposide. FIG. 11C is a graph showing Treatment Naïve SCLC Model NCl-H1048 (Topotecan 0.83 mg/kg/wk, D1-2, q2w clinically equivalent dose per BSA=1×˜1.5 mg/m² Topotecan, q3w, D1-5; 1L Etoposide (25 mg/kg) & Carbo (30 mg/kg) clinically equivalent dose per BSA=1×˜100 mg/m² Etoposide D1-3+ AUC6 Carbo D1, q4w); 11B is a schematic of 1L and 2L treatments. 1L regimen results in similar anti-tumor activity as topotecan treatment at clinically relevant doses (based on BSA/BW calculation). After 3 cycles of 1L treatment, mice were randomized for further 2L treatments.

FIG. 12 is a graph showing that Nal-IRI remains effective in platinum-treated SCLC tumors and is superior to topotecan and irinotecan: 2L SCLC Model: NCl-H1048. In platinum-treated SCLC tumors: Nal-IRI remains active and is trending towards complete response; IRI treatment is active but after 3rd cycle some tumors are trending regrowth; Topotecan (at 2× clinically relevant dose) seems to be active after 1-2 cycles but progress quickly after 3rd dose; Etoposide+carboplatin is not tolerable by the 5th cycle.

FIGS. 13A and 13B are graphs showing that Nal-IRI is also superior to topotecan and irinotecan in another SCLC xenograft model (DMS-114): FIG. 13A is a graph showing DMS-114 SCLC Mouse Xenograft (s.c.); FIG. 13B is a chart showing Nal-IRI (Day 74) tumor volume change. Nal-IRI is superior to irinotecan and topotecan at clinically relevant doses. SCLC tumors respond to irinotecan early on but became less responsive after 2-3 cycles.

FIGS. 14A-4C are graphs showing SCLC tumors treated with TOP1 inhibitors remain responsive to nal-IRI. FIG. 14A. DMS-114: Treatment Naïve; FIG. 14B. DMS-114: Topotecan-Treated; FIG. 14C. DMS-114: Irinotecan-Treated. DMS114 tumors treated with topotecan are responsive to nal-IRI (16 mg/kg) but not irinotecan (33 mg/kg).

FIGS. 15A-15C are graphs showing that duration of exposure maybe crucial for TOP1 inhibitor activity. FIG. 15A is DMS-114 SCLC Mouse Xenograft (s.c.); FIG. 15B is Hypothesized Tumor Exposure; FIG. 15C is NCl-H1048 Mouse Xenograft. At the same dose intensity, bolus (given on day 1) topotecan has less anti-tumor activity compared to fractionated topotecan (days 1 & 2). This may be indicative that prolonged exposure of TOP1 inhibitor above a therapeutic threshold is more beneficial than high Cmax because irinotecan is a pro-drug (CPT-11), the active metabolite SN-38 may also have a longer duration than topotecan.

FIGS. 16A-16D show NCl-H1048 SCLC Mouse Xenograft (s.c.) FIG. 16A. Tumor Volume; FIG. 16B. Survival; FIG. 16C. Body Weight Change; FIG. 16D. Response at Day 98.

FIGS. 17A-7C show NDMC-53 SCLC Mouse Xenograft (s.c.) FIG. 17A. Tumor Volume; FIG. 17B. Survival; FIG. 17C response at Day 98 post inoculation with control, NaI-IRI (16 mg/kg salt) or topotecan (0.83 mg/kg/wk, D1-2)

FIGS. 18A and 18B are graphs showing that Nal-IRI increases exposure and sustains delivery of irinotecan and SN-38 (active metabolite) in BxPC-3 mouse xenograft tumors: FIG. 18A. Plasma; FIG. 18B. Tumor.

FIG. 19 is a graph showing that Nal-IRI effectively delivers irinotecan to tumors in preclinical models of SCLC.

FIGS. 20A and 20B are graphs showing SCLC Tumors treated with TOP1 inhibitors remain responsive to nal-IRI: FIG. 20A. DMS-114: Topotecan-Treated; FIG. 20B. DMS-114: Treatment Naïve. DMS114 tumors treated with topotecan are responsive to nal-IRI (16 mg/kg) but not irinotecan (33 mg/kg).

FIGS. 21A and 21B are graphs showing that Nal-IRI remains effective in platinum-treated SCLC tumors and is superior to topotecan and irinotecan in a 2L SCLC Model: NCl-H1048. FIG. 21A shows the change in Tumor Volume; FIG. 21B is a survival graph.

FIGS. 22A-22D are graphs showing pre-clinical evidence that MM-398 has improved circulation and tumor circulation in a HT29 CRC xenograft model—MM-398 40 mg/kg: FIG. 22A CPT-11 plasma (sustained plasma levels), FIG. 22B. SN-38 plasma (moderately sustained plasma levels), FIG. 22C CPT-11 tumor (sustained intra-tumor levels), and FIG. 22D SN-38 tumor (enhanced intra-tumor activation to SN38).

FIGS. 23A-23F are graphs showing Nal-IRI has greater anti-tumor activity than irinotecan and topotecan. NOD/SCID mice with subcutaneous (FIG. 23A) DMS-53, (FIG. 23B) DMS-114 or (FIG. 23C) NCI-H1048. SCLC xenograft tumors were treated with IV nal-IRI (16 mg/kg; triangles), IV irinotecan (33 mg/kg; diamonds), IP topotecan (0.83 mg/kg/wk days 1-2; squares) or vehicle control (circles). For DMS-114 and NCI-H1048 all groups have n=10; for DMS-53 n=4, 5 and 5 for control, topotecan and nal-IRI, respectively. Balb/c nude mice bearing subcutaneous patient-derived xenografts (FIG. 23D) LUN-182, (FIG. 23E) LUN-081 and (FIG. 24F) LUN-164 were treated with IV nal-IRI (16 mg/kg; triangles), IV irinotecan (33 mg/kg; diamonds), IP topotecan (0.83 mg/kg/wk days 1-2; squares) or vehicle control (circles). For all PDX models n=5 for all groups. Vertical dotted lines indicate start of weekly dosing and error bars indicate standard error of the mean.

### DETAILED DESCRIPTION

MM-398 is a liposomal encapsulation of irinotecan that provides sustained tumor exposure of SN-38 and therefore provides certain advantages over nonliposomal irinotecan. The approved regimen of MM-398 in patients with pancreatic cancer is in combination with 5-FU/LV. However, 5-FU is not an active agent used in the treatment of SCLC. To date, the treatment of patients with SCLC with MM-398 has not been disclosed. Applicants have discovered certain methods and uses of MM-398 monotherapy in patients with SCLC, including the methods and uses disclosed herein.

The discovery of these methods and uses of MM-398 for use in patients with SCLC was based in part on preclinical data and clinical pharmacology analysis described herein. The methods and uses are designed to balance increased efficacy with increased toxicity predicted at higher doses. Preclinical data herein indicate the activity of MM-398 in models of SCLC. Clinical pharmacology analysis supports increased toxicity at increased doses and specifically supports the safety profile of the 90 mg/m² dose. Finally, preclinical efficacy data at mouse dose levels equivalent to 90 mg/m² in humans are shown to be superior to topotecan.

A human patient diagnosed with small cell lung cancer (SCLC) after disease progression following platinum-based therapy for the SCLC can be treated with an antineoplastic therapy consisting of a single dose of a therapeutically effective amount of irinotecan encapsulated in liposomes. The liposomal irinotecan can be a pharmaceutically acceptable liposome formulation of irinotecan, comprising irinotecan in a delivery form having a diameter of about 100 nm, such as an liposomal irinotecan (Example 1), including PEGylated liposomes. Various suitable liposomal irinotecan preparations can be manufactured as disclosed herein (Example 8). Preferably, the liposomal irinotecan is the product MM-398 (ONIVYDE) (Example 9).

As used herein, 90 mg/m² irinotecan refers to the free base, encapsulated in liposomes (dose based on the amount of irinotecan free base) and is equivalent to 100 mg/m² of the irinotecan hydrochloride anhydrous salt). Converting a dose based on irinotecan hydrochloride trihydrate to a dose based on irinotecan free base is accomplished by multiplying the dose based on irinotecan hydrochloride trihydrate with the ratio of the molecular weight of irinotecan free base (586.68 g/mol) and the molecular weight of irinotecan hydrochloride trihydrate (677.19 g/mol). This ratio is 0.87 which can be used as a conversion factor. For example, an 80 mg/m² dose based on irinotecan hydrochloride trihydrate is equivalent to a 69.60 mg/m² dose based on irinotecan free base (80×0.87). In the clinic this is rounded to 70 mg/m² to minimize any potential dosing errors.

Doses of nal-IRI in some studies were calculated based on the equivalent dose of irinotecan hydrochloride trihydrate (salt); in this specification, unless specified otherwise, the doses are based on irinotecan as the free base. Accordingly, 50 mg/m² based on irinotecan as free base is equivalent to 60 mg/m² based on irinotecan as the hydrochloride trihydrate, 70 mg/m² based on irinotecan as free base is equivalent to 80 mg/m² based on irinotecan as the hydrochloride trihydrate, 90 mg/m² based on irinotecan as free base is equivalent to 100 mg/m² based on irinotecan as the hydrochloride trihydrate, and 100 mg/m² based on irinotecan as free base is equivalent to 120 mg/m² based on irinotecan as the hydrochloride trihydrate, in accordance with Table 1.

TABLE 1

| Salt | Free base |
|---|---|
| 180 | 150 |
| 120 | 100 |
| 100 | 90 |
| 80 | 70 |
| 60 | 50 |
| 50 | 45 |
| 40 | 35 |

The pharmacokinetic parameters of total Irinotecan and total SN-38 following administration of MM-398 90 mg/m² as a single agent or part of combination chemotherapy are presented in Table 2.

TABLE 2
Total Irinotecan and Total SN-38 Pharmacokinetic
Parameters in Patients with Solid Tumors.

| | Total Irinotecan | | | Total SN-38 | |
|---|---|---|---|---|---|
| Dose | Cₘₐₓ | AUC_{0-∞} | t_{1/2} | Cₘₐₓ | t_{1/2} |
| (mg/m²) | [μg/mL] | [h · μg/mL] | [h] | [ng/mL] | [h] |
| Max (125%) | 60.5 | 2216.5 | 25.8 | 8.8 | 67.8 |
| 90 | 48.4 | 1773.2 | 25.8 | 7.0 | 67.8 |
| Min (80%) | 38.7 | 1418.6 | 25.8 | 5.6 | 67.8 |

Over the dose range of 50 to 150 mg/m², the Cₘₐₓ and AUC of total irinotecan increases with dose. Additionally, the Cₘₐₓ of total SN-38 increases proportionally with dose; however, the AUC of total SN-38 increases less than proportionally with dose. Higher plasma SN-38 Cₘₐₓ was associated with increased likelihood of experiencing neutropenia.

The Cₘₐₓ of SN-38 increases proportionally with liposomal irinotecan dose but the AUC of SN-38 increases less than proportionally with dose, enabling new methods of dosage adjustment. For example, the value of the parameter associated with adverse effects (Cₘₐₓ) decreases by a relatively greater extent than the value of the parameter associated with the effectiveness of treatment (AUC). Accordingly, when an adverse effect is seen, a reduction in the dosing of the liposomal irinotecan can be implemented that maximizes the difference between the reduction in Cₘₐₓ and in AUC. The discovery means that in treatment regimens, a given SN-38 AUC can be achieved with a surprisingly low SN-38 Cₘₐₓ. Likewise, a given SN-38 Cₘₐₓ can be achieved with a surprisingly high SN-38 AUC.

Direct measurement of irinotecan liposome showed that 95% of irinotecan remains liposome encapsulated, and the ratios between total and encapsulated forms did not change with time from 0 to 169.5 hours post-dose.

In some embodiments, the liposomal irinotecan can be characterized by the parameters in Table 2. In some embodiments, the liposomal irinotecan can be MM-398 or a product that is bioequivalent to MM-398. In some embodiments, the liposomal irinotecan can be characterized by the parameters in Table 3, including a Cₘₐₓ and/or AUC value that is 80-125% of the corresponding value in Table 2. The pharmacokinetic parameters of total irinotecan for various alternative liposomal irinotecan formulations administering 90 mg/m² irinotecan free base once every two weeks is provided in Table 3.

TABLE 3
Total Irinotecan Pharmacokinetic Parameters in
Alternative Liposomal Irinotecan Formulations

Total Irinotecan

| Dose | Cₘₐₓ | AUC_{0-∞} |
|---|---|---|
| (mg/m²) | [μg/mL] (n = 25) | [h · μg/mL] (n = 23) |
| 90 | 38.7-60.5 | 1418.6-2216.5 |

Cₘₐₓ: Maximum plasma concentration
AUC_{0-∞}: Area under the plasma concentration curve extrapolated to time infinity
t_{1/2}: Terminal elimination half-life

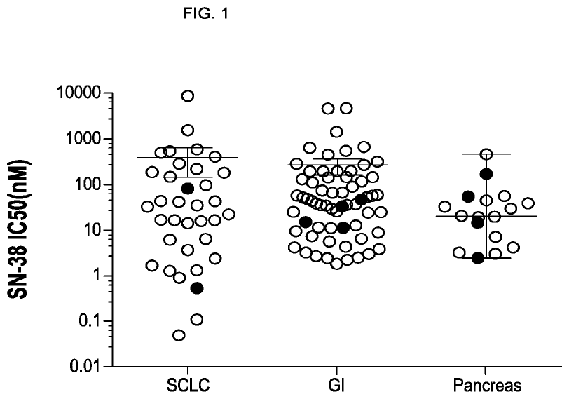

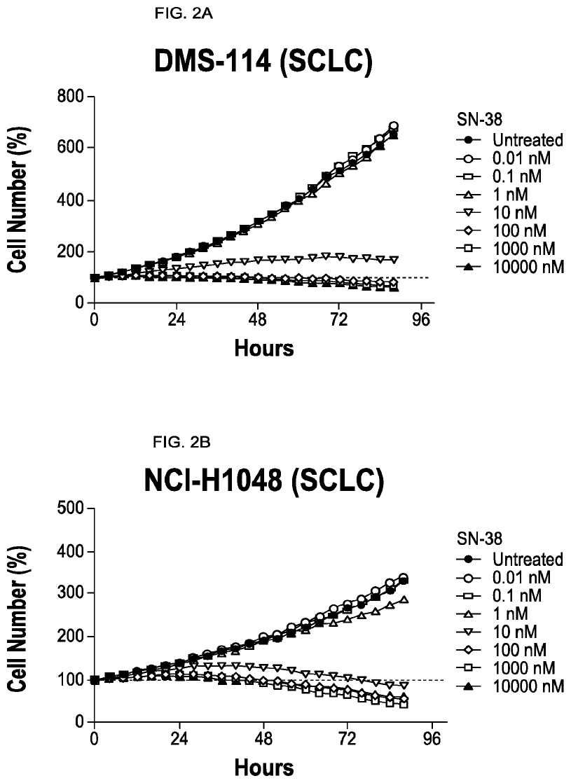

The activity of the active metabolite of irinotecan, SN-38, against various SCLC cell lines was investigated in in vitro growth and viability assays (Example 2). An analysis of this data indicated that SCLC cell lines have similar sensitivity to SN-38 as pancreatic and gastrointestinal cancer cell lines (FIG. 1). In addition, SN-38 induced a decrease in cell viability of >90% in four tested SCLC cell lines, the IC50 was variable and spanned several orders of magnitude. FIGS. 2A and 2B show the cell growth inhibition kinetics of SN-38 in 2 SCLC cell lines, as described in Example 2.

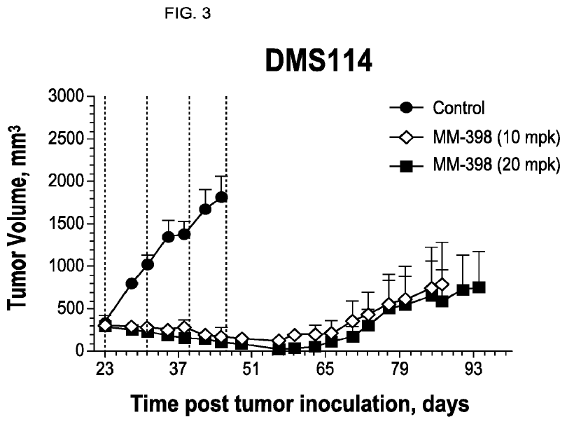

The activity of MM-398 as a single agent was investigated in xenograft models of SCLC (Example 3). As shown in FIG. 3, anti-tumor activity was seen at all dose levels tested in the DMS-114 model.

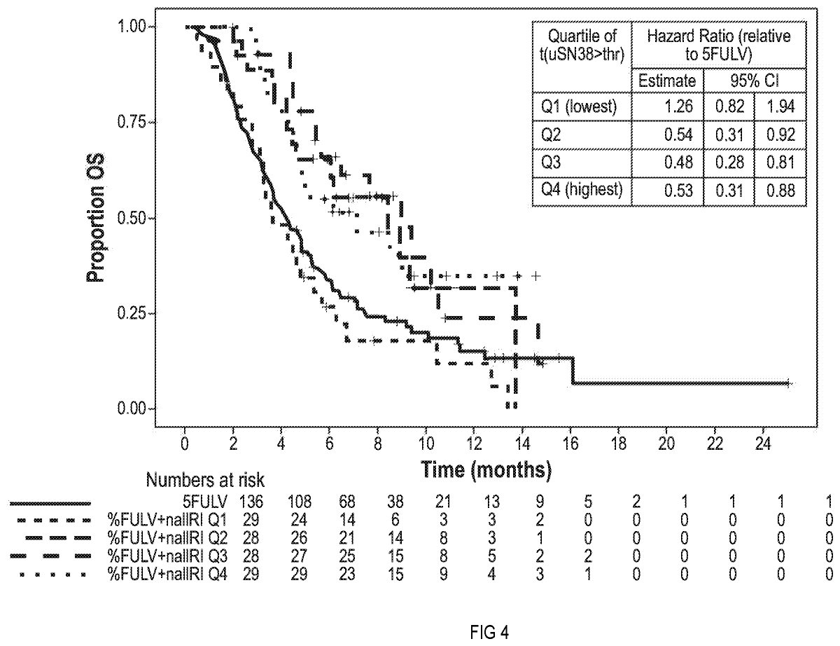

The estimated association between MM-398 exposure and efficacy was evaluated in pancreatic cancer patients (Example 4). The relationship between OS and quartiles of time (uSN38>0.03 ng/mL) for the MM-398+5FU/LV is provided in FIG. 4.

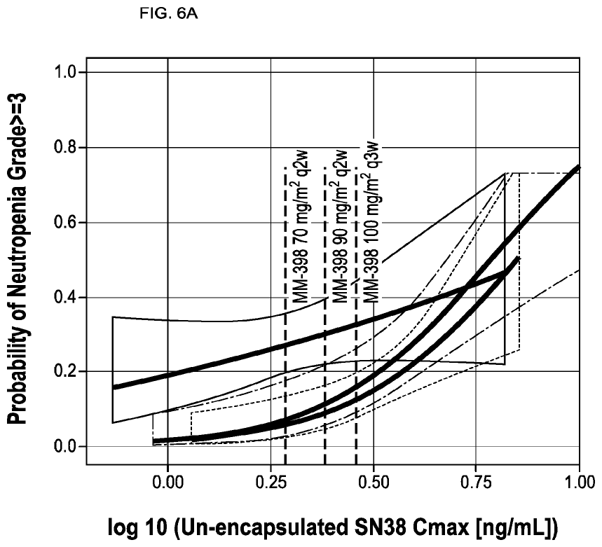

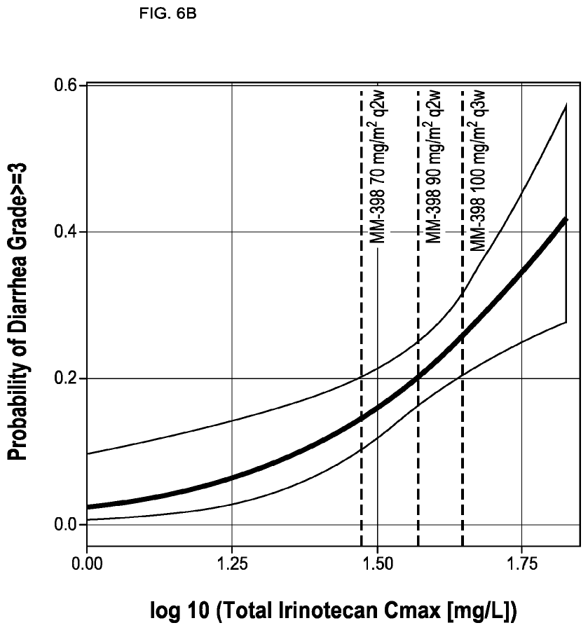

As described in Examples 6 and 7, an antineoplastic therapy consisting of liposomal irinotecan in a pharmaceutically acceptable injectable form can be administered once every two weeks to patients with SCLC disease that has progressed after having received previous antineoplastic therapy (e.g., prior platinum-based therapies alone or with other chemotherapeutic agents). The dose of liposomal irinotecan (e.g., 50-90 mg/m² irinotecan (free base) encapsulated in irinotecan liposomes) and dose frequency (e.g., once every 2 weeks) of liposomal irinotecan can be selected or modified for certain patients. The dose can be selected to provide a tolerable patient dose, including a dose providing acceptably low levels of grade 3 or greater neutropenia (FIG. 6A) and/or diarrhea (FIG. 6B), as described in Example 6. During the antineoplastic therapy, the patient may receive other agents that are not antineoplastic agents, such as anti-emetic agents. The antineoplastic therapy can be administered in the absence of topotecan.

In some embodiments, the invention is a method of treating a human patient diagnosed with small cell lung cancer (SCLC) after disease progression following platinum-based therapy for the SCLC, the method comprising administering to the human patient an antineoplastic therapy once every two weeks, the antineoplastic therapy consisting of a single dose of liposomal irinotecan providing 90 mg/m² (free base) of irinotecan encapsulated in irinotecan liposomes. In some embodiments, the invention is a method of treating a human patient diagnosed with small cell lung cancer (SCLC) after disease progression following platinum-based therapy for the SCLC, the method comprising administering to the human patient an antineoplastic therapy once every two weeks, the antineoplastic therapy consisting of a single dose of liposomal irinotecan providing 70 mg/m² (free base) of irinotecan encapsulated in irinotecan liposomes. In some embodiments, the invention is a method of treating a human patient diagnosed with small cell lung cancer (SCLC) after disease progression following platinum-based therapy for the SCLC, the method comprising administering to the human patient an antineoplastic therapy once every two weeks, the antineoplastic therapy consisting of a single dose of liposomal irinotecan providing 50 mg/m² (free base) of irinotecan encapsulated in irinotecan liposomes.

The methods of treatment can include determining whether a patient meets one or more inclusion criteria specified in Example 7, and then administering the antineoplastic therapy consisting of liposomal irinotecan. For example, an antineoplastic therapy can consist of administering a therapeutically effective dose (e.g., 50-90 mg/m² irinotecan (free base) encapsulated in liposomes) and dose frequency (e.g., once every 2 weeks) of liposomal irinotecan to a patient who has been treated for SCLC with a platinum-based therapy (e.g., cisplatin and/or carboplatin alone or in combination with etoposide).

In addition, the methods of treatment can include determining whether a patient meets one or more exclusion criteria specified in Example 7, and not administering the antineoplastic therapy consisting of liposomal irinotecan. Methods of treating SCLC disclosed herein can include administering the antineoplastic therapy to a patient who does not meet one or more exclusion criteria in Example 7. For example, an antineoplastic therapy can consist of administering a therapeutically effective dose (e.g., 50-90 mg/m² irinotecan (free base) encapsulated in liposomes) and dose frequency (e.g., once every 2 weeks) of liposomal irinotecan to a patient who has not been treated for SCLC with irinotecan or topotecan.

Certain subgroup of patients diagnosed with SCLC may optionally be treated with a reduced dose of the liposomal irinotecan, including patients who have higher levels of bilirubin or patients with UGT1A1*28 7/7 homozygous allele. The reduced dose refers to a dose of less than 90 mg/m² of irinotecan (free base) encapsulated in liposomes administered once every two weeks to the patient receiving the reduced dose. In some examples, the reduced dose can be a dose of 50-90 mg/m², including a reduced dose of 50 mg/m², a reduced dose of 60 mg/m², a reduced dose of 70 mg/m² or a reduced dose of 80 mg/m² irinotecan (free base) administered once every two weeks to patients diagnosed with SCLC and receiving the reduced dose. For those patients who start with 70 mg/m², the first dose reduction should be to 50 mg/m² and then to 43 mg/m². The exact determination of the appropriate dose will be dependent on the observed pharmacokinetics, efficacy, and safety in that subpopulation.

In some examples, the liposomal irinotecan can be administered to patients diagnosed with SCLC disease progression on or after immunotherapy and/or after first-line platinum based chemotherapy (carboplatin or cisplatin) or chemo-radiation including platinum-based chemotherapy for treatment of limited or extensive stage SCLC. In some examples, the patient can receive some form of immunotherapy for SCLC prior to administration of the liposomal irinotecan. Examples of immunotherapy can include atezolizumab, avelimumab, nivolumab, pembrolizumab, ipilimumab, tremelimumab and/or durvalumab. In one example, a patient receives nivolumab for SCLC (e.g., according to a treatment regimen in NCT02481830) prior to receiving the liposomal irinotecan as disclosed herein. In one example, a patient receives ipilimumab for SCLC (e.g., according to a treatment regimen in NCT01331525, NCT02046733, NCT01450761, NCT02538666 or NCT01928394) prior to receiving the liposomal irinotecan as disclosed herein. The immunotherapy can include molecules that bind to CTLA4, PDL1, PD1, 41BB and/or OX40 including the publicly available compounds in the Table 4 below or other compounds that bind to the same epitope or have the same or similar biological functions.

TABLE 4

| Antibody | Antibody Sequence (literature reference) |
|---|---|
| α-PDL1 | 10F.9G2, Bioxcell |
| α -41BB | LOB12.3, Bioxcell |
| α -CTLA4 | 9H10, Bioxcell |
| α -OX40 | OX-86, Bioxcell |

The use of a combination of liposomal irinotecan and an immunotherapy can be used for the treatment of cancer in a host in need thereof, in an amount and in a schedule of administration that is therapeutically synergistic in the treatment of said cancer. The immunotherapy can be an antibody or combination of antibodies binding to and/or acting upon alpha-PDL1, alpha-41BB, alpha-CTLA4, alpha-OX40 and/or PD1.

In some embodiments, the treatment of cancer in a host in need thereof comprises the administration of MM-398 without the administration of steroids.

The treatment schedule can comprise administering MM-398 once every two or three weeks or two out of three weeks at 43, 50, 70, 80 or 90 mg/m² liposomal irinotecan (free base) in combination with an immunotherapy (e.g., in combination with an antibody to alpha-PDL1, PD1, alpha-41BB, alpha-CTLA4 and/or alpha-OX40). For example, the treatment schedule can comprise administering a (e.g., 28-day) treatment cycle to a human host diagnosed with SCLC, where the treatment cycle includes administration of: a total of 43, 50, 70, 80 or 90 mg/m² liposomal irinotecan (free base) followed by the administration of 3 mg/kg nivolumab, once every two weeks; and repeating said treatment cycle until a progression or an unacceptable toxicity is observed. In another example, the treatment schedule can comprise administering a (e.g., 28-day) treatment cycle to a human host diagnosed with SCLC, where the treatment cycle includes administration of: a total of 43, 50, 70, 80 or 90 mg/m² liposomal irinotecan (free base) once every two or three weeks or two out of three weeks, followed by the administration of 2 mg/kg pembrolizumab, once every two or three weeks (where the first dose of liposomal irinotecan and pembrolizumab are given on the same day); and repeating said treatment cycle until a progression or an unacceptable toxicity is observed. The treatment schedule can comprise administering MM-398 once every two weeks at 90 mg/m² liposomal irinotecan (free base).

A method of treating a human patient diagnosed with small cell lung cancer (SCLC) after disease progression following platinum-based therapy for the SCLC, can consist of administering to the human patient an antineoplastic therapy once every two weeks, the antineoplastic therapy consisting of a single dose of liposomal irinotecan providing 50, 70 or 90 mg/m² (free base) of irinotecan encapsulated in irinotecan liposomes. When the patient is known to be homozygous for the UGT1A1*28 allele, each dose of irinotecan liposome can be reduced (e.g., 50, or 70 mg/m²). When the patient is not homozygous for the UGT1A1*28 allele, and is not otherwise reduced, each dose of irinotecan liposome can be 90 mg/m². The method can further comprise administering a corticosteroid and an anti-emetic to the patient prior to the administration of the irinotecan liposome.

A method of treating a human patient not homozygous for the UGT1A1*28 allele and diagnosed with small cell lung cancer (SCLC) after disease progression following prior therapy for the SCLC, can comprise administering to the human patient an antineoplastic therapy once every two weeks, the antineoplastic therapy consisting of a single dose of liposomal irinotecan providing 90 mg/m² of irinotecan (free base) encapsulated in a irinotecan liposomes. The method can further comprise administering a corticosteroid and an anti-emetic to the patient prior to the administration of the irinotecan liposome.

Prior to receiving the antineoplastic therapy of liposomal irinotecan, the patient can be a patient who has progressed on a platinum-based regimen and who has also (optionally) received a single line of immunotherapy either in maintenance or 2L setting. The patient can be a patient who was not treated with topotecan for the SCLC prior to receiving the liposomal irinotecan antineoplastic therapy. The patient can previously receive immunotherapy induction, followed and/or accompanied by one or more maintenance doses of chemotherapy, prior to administration of the liposomal irinotecan.

The treatment schedule can comprise administering MM-398 once every three weeks at 100-130 mg/m² liposomal irinotecan (free base) in combination with an immunotherapy (e.g., in combination with an antibody to alpha-PDL1, PD1, alpha-41BB, alpha-CTLA4 and/or alpha-OX40). For example, the treatment schedule can comprise administering a treatment cycle to a human host diagnosed with SCLC, where the treatment cycle includes administration of: a total of 100, 110, 120, or 130 mg/m² liposomal irinotecan (free base) followed by the administration of 3 mg/kg nivolumab, once every three weeks; and repeating said treatment cycle until a progression or an unacceptable toxicity is observed. The treatment schedule can comprise administering a treatment cycle to a human host diagnosed with SCLC, where the treatment cycle includes administration of: a total of 100, 110, 120, or 130 mg/m² liposomal irinotecan (free base) once every three weeks combined with the administration of 3 mg/kg nivolumab, once every two or three weeks (where the first dose of liposomal irinotecan and nivolumab are given on the same day); and repeating said treatment cycle until a progression or an unacceptable toxicity is observed. In another example, the treatment schedule can comprise administering a treatment cycle to a human host diagnosed with SCLC, where the treatment cycle includes administration of: a total of 100, 110, 120, or 130 mg/m² liposomal irinotecan (free base) followed by the administration of 2 mg/kg pembrolizumab, once every three weeks; and repeating said treatment cycle until a progression or an unacceptable toxicity is observed. The treatment schedule can comprise administering a treatment cycle to a human host diagnosed with SCLC, where the treatment cycle includes administration of: a total of 100, 110, 120, or 130 mg/m² liposomal irinotecan (free base) once every three weeks combined with the administration of 2 mg/kg pembrolizumab, once every two or three weeks (where the first dose of liposomal irinotecan and pembrolizumab are given on the same day); and repeating said treatment cycle until a progression or an unacceptable toxicity is observed. The treatment schedule can comprise administering a treatment cycle to a human host diagnosed with SCLC, where the treatment cycle includes administration of: a total of 100, 110, 120, or 130 mg/m² liposomal irinotecan (free base) once every two out of three weeks combined with the administration of 2 mg/kg pembrolizumab, once every two or three weeks (where the first dose of liposomal irinotecan and pembrolizumab are given on the same day); and repeating said treatment cycle until a progression or an unacceptable toxicity is observed. The treatment schedule can comprise administering MM-398 once every three weeks at 110 mg/m² liposomal irinotecan (free base), in combination with a therapeutically effective amount of an immunotherapy (e.g., in combination with an antibody to alpha-PDL1, PD1, alpha-41BB, alpha-CTLA4 and/or alpha-OX40). The treatment schedule can comprise administering MM-398 once every three weeks at 100 mg/m² liposomal irinotecan (free base), in combination with a therapeutically effective amount of an immunotherapy (e.g., in combination with an antibody to alpha-PDL1, PD1, alpha-41BB, alpha-CTLA4 and/or alpha-OX40). The treatment schedule can comprise administering MM-398 once every three weeks at 120 mg/m² liposomal irinotecan (free base), in combination with a therapeutically effective amount of an immunotherapy (e.g., in combination with an antibody to alpha-PDL1, PD1, alpha-41BB, alpha-CTLA4 and/or alpha-OX40). The treatment schedule can comprise administering MM-398 once every three weeks at 130 mg/m² liposomal irinotecan (free base), in combination with a therapeutically effective amount of an immunotherapy (e.g., in combination with an antibody to alpha-PDL1, PD1, alpha-41BB, alpha-CTLA4 and/or alpha-OX40).

In some embodiments, liposomal irinotecan is administered after disease progression following platinum-based therapy for the SCLC in combination with one or more of prexasertib, aldoxorubicin, lurbinectedin and Rova-T. In some embodiments the liposomal irinotecan can be administered to a patient who has previously received a PD-1 directed therapeutic (e.g., nivolumab, pembrolizumab), a PD-L1 directed therapeutic (e.g., atezolizumab or durvalumab), or a Notch ADC compound (e.g., Rova-T) as a first-line (1L) therapy for the SCLC. In some embodiments the liposomal irinotecan can be administered in combination with a Chk1 directed therapeutic (e.g., prexasertib), a Topo-2 directed therapeutic (e.g., aldozurubicin), a DNA inhibitor (e.g., lurbinectedin) or a Notch ADC compound (e.g., Rova-T). In other embodiments the liposomal irinotecan can be administered in the absence (i.e., without) a Chk1 directed therapeutic (e.g., prexasertib), a Topo-2 directed therapeutic (e.g., aldozurubicin), a DNA inhibitor (e.g., lurbinectedin) or a Notch ADC compound (e.g., Rova-T). In some embodiments the liposomal irinotecan can be administered to a patient who has previously received cisplatin or carboplatin for SCLC, and the liposomal irinotecan is administered in the absence of (i.e., without) cisplatin or carboplatin (for second or subsequent lines of therapy).

In some embodiments, methods of treating SCLC can comprise administering a treatment cycle to a human host diagnosed with SCLC, where the treatment cycle includes administration of: a total of 90 mg/m² liposomal irinotecan (free base) or 120 mg/m² liposomal irinotecan (free base) once every three weeks combined with the administration of 3 mg/kg nivolumab once every two weeks starting on the same day as the first administration of the liposomal irinotecan, and repeating said treatment cycle until a progression or an unacceptable toxicity is observed. In another example, the treatment schedule can comprise administering a treatment cycle to a human host diagnosed with SCLC, where the treatment cycle includes administration of: a total of 90 mg/m² liposomal irinotecan (free base) or 120 mg/m² liposomal irinotecan (free base) once every three weeks combined with the administration of 2 mg/kg pembrolizumab once every three weeks starting on the same day as the first administration of the liposomal irinotecan; and repeating said treatment cycle until a progression or an unacceptable toxicity is observed.

The patient can be administered antineoplastic therapy for treatment of SCLC comprising 90 mg/m² liposomal irinotecan once every two weeks, without the administration of another antineoplastic agent (e.g., without the administration of topotecan).

Preferably, the antineoplastic therapy for previously treated (e.g. second line) SCLC provides a median time to progression of progression free survival of greater than 15 weeks (e.g., at least about 20-25 weeks, including about 21-24 weeks, about 22-24 weeks, about 23 weeks or about 24 weeks), a median overall survival of greater than 30 weeks (e.g., at least about 30-50 weeks, including about 40-50 weeks, about 44-48 weeks, about 45-47 weeks, about 46 weeks or about 47 weeks), with a hazard ratio of less than 1, and preferably less than 0.7, 0.6 or 0.5 (e.g., including hazard ratio of about 0.6-0.7). Preferably, the antineoplastic therapy provides a major adverse event (grade 3+) occurring in >5% of the population of less than 50% for neutropenia (e.g., about 10-50%, including about 20%), less than 50% for thrombocytopenia (e.g., less than 10%, including 1-10%, 1-5%, less than 5%, and about 2%, about 3% and about 4%), and less than 30% for anemia (e.g., less than 10%, including 1-10%, 1-8%, less than 8%, and about 5-7%, about 6% and about 5%).

A method of treating a human patient diagnosed with small cell lung cancer (SCLC) after disease progression following platinum-based therapy for the SCLC, can consist of administering to the human patient an antineoplastic therapy once every two weeks, the antineoplastic therapy consisting of a single dose of liposomal irinotecan providing 90 mg/m² (free base) of irinotecan encapsulated in irinotecan liposomes (or reduced doses of 50-70 g/m² (free base) of irinotecan as the liposomal irinotecan to patients who have experienced adverse events during or after a prior administration of liposomal irinotecan and/or patients known to be homozygous for the UGT1A1*28 allele), where the antineoplastic therapy in a clinical trial of at least 300 patients (e.g., about 400-450 patients), where the antineoplastic therapy in a clinical trial of at least 300 patients (e.g., about 400-450 patients) results in major adverse event (grade 3+) occurring in >5% of the population of less than 50% for neutropenia (e.g., about 10-50%, including about 20%), less than 50% for thrombocytopenia (e.g., less than 10%, including 1-10%, 1-5%, less than 5%, and about 2%, about 3% and about 4%), and less than 30% for anemia (e.g., less than 10%, including 1-10%, 1-8%, less than 8%, and about 5-7%, about 6% and about 5%).

A method of treating a human patient diagnosed with small cell lung cancer (SCLC) after disease progression following platinum-based therapy for the SCLC, can consist of administering to the human patient an antineoplastic therapy once every two weeks, the antineoplastic therapy consisting of a single dose of liposomal irinotecan providing 90 mg/m² (free base) of irinotecan encapsulated in irinotecan liposomes (or reduced doses of 50-70 g/m² (free base) of irinotecan as the liposomal irinotecan to patients who have experienced adverse events during or after a prior administration of liposomal irinotecan and/or patients known to be homozygous for the UGT1A1*28 allele), where the antineoplastic therapy in a clinical trial of at least 300 patients (e.g., about 400-450 patients) results in one or more of the following: median time to progression of progression free survival of greater than 15 weeks (e.g., at least about 20-25 weeks, including about 21-24 weeks, about 22-24 weeks, about 23 weeks or about 24 weeks), a median overall survival of greater than 30 weeks (e.g., at least about 30-50 weeks, including about 40-50 weeks, about 44-48 weeks, about 45-47 weeks, about 46 weeks or about 47 weeks), with a hazard ratio of less than 1, and preferably less than 0.7, 0.6 or 0.5 (e.g., including hazard ratio of about 0.6-0.7).

When the patient is known to be homozygous for the UGT1A1*28 allele, each dose of irinotecan liposome can be reduced (e.g., 50, or 70 mg/m²). When the patient is not homozygous for the UGT1A1*28 allele, and is not otherwise reduced, each dose of irinotecan liposome can be 90 mg/m². The method can further comprise administering a corticosteroid and an anti-emetic to the patient prior to the administration of the irinotecan liposome.

In some embodiments, the liposomal irinotecan can be administered to patients diagnosed with small cell lung cancer (SCLC) disease progression following treatment with one or more camptothecin compounds or topoisomerase I (Topo-1) inhibitors. Examples of camptothecin compounds or topoisomerase I (Topo-1) inhibitors include, but are not limited to, camptothecin, 9-aminocamptothecin, 7-ethylcamptothecin, 10-hydroxycamptothecin, 7-ethyl 10-hydroxy camptothecin, 9-nitrocamptothecin, 10,11-methylenedioxycamptothecin, 9-amino-10,11-methylenedioxycamptothecin, 9-chloro-10,11-methylenedioxycamptothecin, irinotecan (CPT-11), topotecan, lurtotecan, silatecan, etirinotecan pegol, rubitecan, exatecan, FL118, belotecan, gimatecan, indotecan, indimitecan, (7-(4-methylpiperazinomethylene)-10,11-ethylenedioxy-20(S)-camptothecin, 7-(4-methylpiperazinomethylene)-10,11-methylenedioxy-20(S)-camptothecin, and 7-(2-N-isopropylamino)ethyl)-(20S)-camptothecin.

In some embodiments the liposomal irinotecan can be administered to patients diagnosed with SCLC disease progression following treatment with irinotecan (CPT-11), topotecan, or both. In some embodiments the liposomal irinotecan can be administered to patients diagnosed with SCLC disease progression following treatment with irinotecan (CPT-11). In some embodiments the liposomal irinotecan can be administered to patients diagnosed with SCLC disease progression following treatment with topotecan. In some embodiments the liposomal irinotecan can be administered to patients diagnosed with SCLC disease progression following treatment with non-liposomal irinotecan.

In some embodiments, the platinum-based therapy is administered in combination with etoposide or non-liposomal irinotecan. In some embodiments, the platinum-based therapy is administered in combination with etoposide. In some embodiments, the platinum-based therapy is administered in combination with non-liposomal irinotecan. One embodiment is a method of treating a human patient diagnosed with small cell lung cancer (SCLC) following disease progression on or after camptothecin-based therapy for the SCLC, the method comprising administering to the human patient an antineoplastic therapy once every two weeks, the antineoplastic therapy consisting of a 90 mg/m² (free base) dose of MM-398 liposomal irinotecan. In some embodiments, the camptothecin-based therapy comprises the prior, discontinued administration of topotecan or non-liposomal irinotecan to treat the human patient diagnosed with SCLC. In some embodiments, the camptothecin-based therapy comprises the prior, discontinued administration of non-liposomal irinotecan administered to the human patient at a 300 mg/m² dose once every three weeks. In some embodiments, the camptothecin-based therapy comprises the prior, discontinued administration of non-liposomal irinotecan administered to the human patient at a 1.5 mg/m² dose of topotecan on days 1, 2, 3, 4, and 5 in a three week treatment cycle.

In some embodiments, the human patient diagnosed with SCLC is platinum sensitive. In some embodiments the human patient diagnosed with SCLC is platinum resistant.

A first aspect of the present disclosure is a method of treating a human patient diagnosed with small cell lung cancer (SCLC) following disease progression on or after first-line platinum-based therapy for the SCLC. One embodiment of the first aspect is a method of treating a human patient diagnosed with small cell lung cancer (SCLC) following disease progression on or after first-line platinum-based therapy for the SCLC, the method comprising administering to the human patient an antineoplastic therapy once every two weeks, the antineoplastic therapy consisting of a 90 mg/m2 (free base) dose of MM-398 liposomal irinotecan.

In one embodiment of the first aspect the platinum-based therapy comprises the prior, discontinued administration of cisplatin or carboplatin to treat the human patient diagnosed with SCLC. In another embodiment the human patient has a blood ANC greater than 1,500 cells/microliter without the use of hematopoietic growth factors, prior to the administration of the MM-398 liposomal irinotecan. Another embodiment is a method of treating a human patient diagnosed with small cell lung cancer (SCLC) following disease progression on or after first-line platinum-based therapy for the SCLC. Yet another embodiment is a method of treating a human patient diagnosed with small cell lung cancer (SCLC) following disease progression on or after first-line platinum-based therapy for the SCLC, the method comprising administering to the human patient an antineoplastic therapy once every two weeks, the antineoplastic therapy consisting of a 90 mg/m2 (free base) dose of MM-398 liposomal irinotecan wherein the human patient has a blood platelet count of greater than 100,000 cells per microliter, prior to the administration of the MM-398 liposomal irinotecan.

In some embodiments of the first aspect the human patient has a blood hemoglobin greater than 9 g/dL, prior to the administration of the MM-398 liposomal irinotecan. In some embodiments the human patient has a serum creatinine of less than or equal to 1.5×ULN and a creatinine clearance of greater than or equal to 40 mL/min prior to the administration of the MM-398 liposomal irinotecan.

In some of the embodiments of the first aspect the human patient has not received a topoisomerase I inhibitor prior to administration of the MM-398 liposomal irinotecan. In yet other embodiments of the first aspect the human patient has not received more than a single platinum-based therapy prior to administration of the MM-398 liposomal irinotecan.

Embodiments of the first aspect may comprise a method wherein the antineoplastic therapy comprises the steps of: (a) preparing a pharmaceutically acceptable injectable composition by combining dispersion of MM-398 liposomal irinotecan containing 4.3 mg irinotecan free base/mL of the dispersion with a 5% Dextrose Injection (D5W) or 0.9% Sodium Chloride Injection to obtain the injectable composition having a final volume of 500 mL and 90 mg/m² (free base) of the MM-398 liposomal irinotecan (±5%); and (b) administering the injectable composition from step (a) containing the MM-398 irinotecan liposome to the patient in a 90-minute infusion.

In any embodiment of the first aspect the method may further comprise administering to the human patient dexamethasone and a 5-HT3 blocker prior to each administration of the antineoplastic therapy, and optionally further administering an antiemetic to the human patient.

A second aspect of the present disclosure is a method of treating a human patient who is not homozygous for the UTG1A1*28 allele and is diagnosed with small cell lung cancer (SCLC) following disease progression on or after first-line platinum-based therapy for the SCLC. One embodiment of the second aspect is a method of treating a human patient who is not homozygous for the UTG1A1*28 allele and is diagnosed with small cell lung cancer (SCLC) following disease progression on or after first-line platinum-based therapy for the SCLC, the method comprising administering to the human patient an antineoplastic therapy once every two weeks in a six-week cycle, the antineoplastic therapy consisting of a 90 mg/m2 (free base) dose of MM-398 liposomal irinotecan.

In some embodiments of the second aspect the platinum-based therapy comprises the prior, discontinued administration of cisplatin or carboplatin to treat the human patient diagnosed with SCLC.

One embodiment of the second aspect is a method of treating a human patient who is not homozygous for the UTG1A1*28 allele and is diagnosed with small cell lung cancer (SCLC) following disease progression on or after first-line platinum-based therapy for the SCLC, wherein the method comprises administering to the human patient an antineoplastic therapy once every two weeks in a six-week cycle, the antineoplastic therapy consisting of a 90 mg/m2 (free base) dose of MM-398 liposomal irinotecan, wherein the human patient has one or more of the following prior to the administration of the MM-398 liposomal irinotecan: (a) a blood ANC greater than 1,500 cells/microliter without the use of hematopoietic growth factors; (b) a blood platelet count of greater than 100,000 cells per microliter; (c) a blood hemoglobin greater than 9 g/dL; and (d) a serum creatinine of less than or equal to 1.5×ULN and a creatinine clearance of greater than or equal to 40 mL/min.

In some embodiments of the second aspect the human patient has not received a topoisomerase I inhibitor prior to administration of the MM-398 liposomal irinotecan; and the human patient has not received a more than a single platinum-based therapy prior to administration of the MM-398 liposomal irinotecan. In some embodiments the method comprises administering the antineoplastic therapy for at least three six-week cycles.

In some embodiments of the second aspect the antineoplastic therapy comprises the steps of: (a) preparing a pharmaceutically acceptable injectable composition by combining dispersion of MM-398 liposomal irinotecan containing 4.3 mg irinotecan free base/mL of the dispersion with a 5% Dextrose Injection (D5W) or 0.9% Sodium Chloride Injection to obtain the injectable composition having a final volume of 500 mL and 90 mg/m2 (free base) of the MM-398 liposomal irinotecan (±5%); and (b) administering the injectable composition from step (a) containing the MM-398 irinotecan liposome to the patient in a 90-minute infusion. This embodiment may further comprise administering to the human patient dexamethasone and a 5-HT3 blocker prior to each administration of the antineoplastic therapy, and optionally further administering an antiemetic to the human patient.

A third aspect of the disclosure provides methods of treating a human patient diagnosed with small cell lung cancer (SCLC) following disease progression on or after a first-line platinum-based therapy for the SCLC selected from the group consisting of cisplatin or carboplatin. One embodiment of the third aspect is a method of treating a human patient diagnosed with small cell lung cancer (SCLC) following disease progression on or after a first-line platinum-based therapy for the SCLC selected from the group consisting of cisplatin or carboplatin. the method comprising administering to the human patient an antineoplastic therapy once every two weeks for a total of at least three six-week cycles, the antineoplastic therapy consisting of a 90 mg/m2 (free base) dose of MM-398 liposomal irinotecan; wherein the human patient is not homozygous for the UTG1A1*28 allele and has the following prior to the administration of each antineoplastic therapy of MM-398 liposomal irinotecan: (a) a blood ANC greater than 1,500 cells/microliter without the use of hematopoietic growth factors; (b) a blood platelet count of greater than 100,000 cells per microliter; (c) a blood hemoglobin greater than 9 g/dL; and (d) a serum creatinine of less than or equal to 1.5×ULN and a creatinine clearance of greater than or equal to 40 mL/min. In some embodiments of the third aspect the human patient has not received a topoisomerase I inhibitor prior to administration of the MM-398 liposomal irinotecan and has not received a more than a single platinum-based therapy prior to administration of the MM-398 liposomal irinotecan; and the method further comprises administering to the human patient dexamethasone and a 5-HT3 blocker prior to each administration of the antineoplastic therapy, and optionally further administering an antiemetic to the human patient.

In one embodiment of the third aspect the antineoplastic therapy comprises the steps of: (a) preparing a pharmaceutically acceptable injectable composition by combining dispersion of MM-398 liposomal irinotecan containing 4.3 mg irinotecan free base/mL of the dispersion with a 5% Dextrose Injection (D5W) or 0.9% Sodium Chloride Injection to obtain the injectable composition having a final volume of 500 mL and 90 mg/m2 (free base) of the MM-398 liposomal irinotecan (±5%); and (b) administering the injectable composition from step (a) containing the MM-398 irinotecan liposome to the patient in a 90-minute infusion.

### EXAMPLES

### Example 1: Liposomal Irinotecan

The liposomal irinotecan composition preferably comprises or consists of phosphatidylcholine, cholesterol, and a polyethyleneglycol-derivatized phosphatidyl-ethanolamine. The liposomal irinotecan can include unilamellar lipid bilayer vesicles comprising the phosphatidylcholine and cholesterol, encapsulating irinotecan sucrose octasulfate. The irinotecan liposomes in the liposomal irinotecan composition have a diameter of 110 nm (±20%). The liposomal irinotecan can comprise irinotecan sucrose octasulfate encapsulated in liposomes having a unilamellar lipid bilayer vesicle, approximately 110 nm in diameter, which encapsulates an aqueous space containing irinotecan in a gelated or precipitated state as the sucrose octasulfate salt; wherein the vesicle is composed of 1,2-distearoyl-sn-glycero-3-phosphocholine (DSPC) (e.g., about 6.8 mg/mL), cholesterol (e.g., about 2.2 mg/mL), and methoxy-terminated polyethylene glycol (MW 2000)-distearoylphosphatidyl ethanolamine (MPEG-2000-DSPE) (e.g., about 0.1 mg/mL). Each mL also contains 2-[4-(2-hydroxyethyl) piperazin-1-yl]ethanesulfonic acid (HEPES) as a buffer (e.g., about 4.1 mg/mL) and sodium chloride as an isotonicity reagent (e.g., about 8.4 mg/mL).

The lipid membrane of the liposomal irinotecan can be composed of phosphatidylcholine, cholesterol, and a polyethyleneglycol-derivatized phosphatidyl-ethanolamine in a suitable molar ratio (e.g., of about 3:2:0.015, and/or in the amount of approximately one polyethyleneglycol (PEG) molecule for 200 phospholipid molecules). ONIVYDE® (also referred to herein as MM-398 or nal-IRI) is a preferred liposomal irinotecan, comprising small unilamellar lipid bilayer vesicle (SUV), approximately 110 nm in diameter that encapsulates an aqueous space which contains irinotecan in a gelated or precipitated state as the sucrosofate salt. The ONIVYDE liposomal irinotecan comprises irinotecan sucrose octasulfate encapsulated in liposomes having a unilamellar lipid bilayer vesicle, approximately 110 nm in diameter, which encapsulates an aqueous space containing irinotecan in a gelated or precipitated state as the sucrose octasulfate salt; wherein the vesicle is composed of 1,2-distearoyl-sn-glycero-3-phosphocholine (DSPC) (6.8 mg/mL), cholesterol (2.2 mg/mL), and methoxy-terminated polyethylene glycol (MW 2000)-distearoylphosphatidyl ethanolamine (MPEG-2000-DSPE) (0.1 mg/mL). Each mL also contains 2-[4-(2-hydroxyethyl) piperazin-1-yl]ethanesulfonic acid (HEPES) as a buffer (4.1 mg/mL) and sodium chloride as an isotonicity reagent (8.4 mg/mL). ONIVYDE is a sterile, white to slightly yellow opaque isotonic liposomal dispersion.

The liposomal irinotecan can be supplied as a sterile, white to slightly yellow, opaque liposomal dispersion in a 10 mL single use glass vial, containing 43 mg/10 mL irinotecan free base. The liposomal dispersion in the vial can be diluted prior to intravenous infusion over 90 minutes.

The present disclosure provides for use of liposomal irinotecan (e.g., ONIVYDE described in Example 9) for the treatment of SCLC once every two weeks at a total dose of 90 mg/m² irinotecan (free base), encapsulated in liposomes (dose based on the amount of irinotecan free base; equivalent to 100 mg/m² of the irinotecan hydrochloride anhydrous salt) IV over 90 minutes, every 2 weeks (preferably, in a 6-week cycle). The recommended starting dose of ONIVYDE in patients known to be homozygous for the UGT1A1*28 allele is 50 mg/m² (free base) administered by intravenous infusion over 90 minutes. The dose of ONIVYDE may be increased to 70 mg/m² as tolerated in subsequent cycles. There is no recommended dose of ONIVYDE for patients with serum bilirubin above the upper limit of normal.

### Example 2

Topoisomerase I inhibition has potent effects on a wide range of cancer cell lines. Reference data in the Wellcome Trust Sanger Institute database of the “Genomics of Drug Sensitivity in Cancer” project are available for 663 cancer cell lines screened for sensitivity to SN-38 (URL www.cancerrxgene.org/translation/Drug/1003). An analysis of this data indicated that SCLC cell lines have similar sensitivity to SN-38 as pancreatic and gastrointestinal cancer cell lines (FIG. 1). Within this dataset, cancer cell lines of gastrointestinal (HT-29, HCT-116, LoVo, MKN45) or pancreatic (AsPC-1, BxPC3, CFPAC-1, MiaPaCa-2) origin for which significant in vivo anti-tumor efficacies of MM-398 have been observed are highlighted by filled circles. SCLC cell lines DMS114 and NCI-H1048 (see below) are also shown as filled circles.

The activity of the active metabolite of irinotecan, SN-38, against various SCLC cell lines was investigated in in vitro growth and viability assays. SN-38 induced a decrease in cell viability of >90% in four tested SCLC cell lines (DMS53, DMS114, NCI-H1048, SW1271), the IC50 was variable and spanned several orders of magnitude. FIGS. 2A and 2B show the cell growth inhibition kinetics of SN-38 in 2 SCLC cell lines (DMS-114 and NCI-H1048) using an IncuCyte® ZOOM System over a time-course of 88 hours. Effective cell growth inhibition was observed between 1-10 nM, while cell killing was observed after prolonged incubation times at concentrations ≥10 nM. This range of SN-38 therapeutic threshold coincides with the amount of SN-38 measured from patient tumor biopsies at 72h post-MM-398 administration (range: 3-163 nM). These data suggest that the prolonged duration of SN-38 in tumors as a result of MM-398 pharmacological characteristics would provide effective activity in SCLC. Preclinical experiments have demonstrated that MM-398 greatly increased the availability of SN-38 in the tumor and showed dose-dependent anti-tumor efficacy at much lower doses than non-liposomal irinotecan.

### Example 3

The activity of MM-398 as a single agent was investigated in xenograft models of SCLC. DMS114 cells were inoculated subcutaneously in NCR nu/nu mice. When tumors reached ˜300 mm³ in volume, mice were treated with 10 or 20 mg/kg of MM-398 irinotecan hydrochloride, administered intravenously on a weekly basis for 4 weeks. Dose levels were selected to correspond to what is believed to be the clinically relevant mouse dose, based on PK modeling and comparison with clinical PK data. As shown in FIG. 3, anti-tumor activity was seen at all dose levels tested in the DMS114 model. Animals with tumors receiving 10 or 20 mg/kg showed tumor regression that was sustained for approximately 20-27 days past the last dose of MM-398 (⅖ and ⅘ complete regressions at 10 and 20 mg/kg dose, respectively).

### Example 4: Association Between Exposure and Efficacy

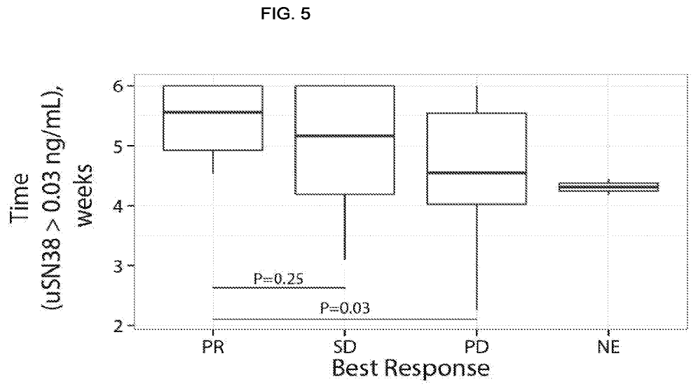

While the association between MM-398 exposure and efficacy is to be studied in SCLC, data analysis in pancreatic cancer patients indicates benefits in increased exposure to SN-38. In the MM-398+5FU/LV treatment arm of NAPOLI-1, longer overall survival (OS) and progression free survival (PFS) were associated with longer time uSN38>0.03 ng/mL and higher Cavg of tIRI, tSN38 and uSN38, with the highest association observed for the time when uSN38>0.03 ng/mL. Cₘₐₓ of tIRI, tSN38, or uSN38 was not predictive of OS (P=0.81-0.92). The relationship between OS and quartiles of time (uSN38>0.03 ng/mL) for the MM-398+5FU/LV is provided in FIG. 4. Longer duration of uSN38>0.03 ng/mL was associated with a higher probability of achieving objective response in the MM-398+5FU/LV arm (FIG. 5) This association was not observed in the MM-398 monotherapy arm dosed at 100 mg/m2 every 3 weeks (P=0.62). The lack of association in the monotherapy arm may be attributed partly in the difference of the dose intervals (MM-398 dose in the monotherapy arm is 100 mg/m2 every 3 weeks, MM-398 dose in the MM-398+5-FU/LV arm is 70 mg/m2 every 2 weeks).

### Example 5: Association Between Exposure and Safety with MM-398

The association between exposure and safety was evaluated based on data in 353 patients treated with Onivyde. Higher un-encapsulated SN-38 Cₘₐₓ was associated with higher probability of both incidence and severity of neutropenia treatment-emergent adverse events (FIG. 6A). Higher total irinotecan Cₘₐₓ was associated with higher probability of observing grade 3+ diarrhea (FIG. 6B). Moreover, different probabilities of observing grade 3+ neutropenia were seen with and without co-administration with 5FU/LV. These associations were used to evaluate the predicted safety with alternative dose regimens to be tested in SCLC.

### Example 6: Prediction of Safety for a Dose of 90 mg/m²

Based on these exposure-safety associations for neutropenia (FIG. 6A) and diarrhea (FIG. 6B), the predicted rate of grade 3+ neutropenia and diarrhea is provided in Table 5. Compared to a dose of 70 mg/m² (free base) as monotherapy, a dose of 90 mg/m² (free base) is predicted to increase grade 3+ neutropenia from 8.4% to 11.1% and diarrhea from 14.3% to 20.0%. These rates were derived based on data with the majority (73%) of patients with pancreatic cancer disease who may have higher risk of diarrhea as compared to patients with SCLC.

TABLE 5
Predicted neutropenia and diarrhea grade 3 or
Higher by Irinotecan Liposome Injection Dose

| Adverse Event code | Dose (mg/m2) | Predicted rates |
|---|---|---|
| Neutropenia | 70 | 8.4% |
| grade >= 3 | 90 | 11.1% |
| | 100 | 13.9% |
| Diarrhea | 70 | 14.3% |
| grade >=3 | 90 | 20.0% |
| | 100 | 25.8% |

### Example 7

A Randomized, Open Label Phase 3 Study of nal-IRI (ONIVYDE® or MM-398) in Patients with Small Cell Lung Cancer Who have Progressed On or After

Platinum-based First-Line Therapy

Overview of Study Design. This is an open label, randomized Phase 3 study of irinotecan liposome injection versus IV topotecan in patients with small cell lung cancer who have progressed on or after platinum-based first line therapy. The study will be conducted in two parts.

Part 1:

Part 1a The objectives of Part 1a are to: 1) describe the safety and tolerability of irinotecan liposome injection monotherapy administered every 2 weeks and 2) to determine the irinotecan liposome injection monotherapy dose (90 mg/m² or 70 mg/m² administered every two weeks) for the Part 1b and Part 2 of this study.

Part 1b is a parallel study of nal-IRI (N=25) and IV topotecan (N=25) for the purpose of characterizing preliminary efficacy and safety of irinotecan liposome injection and IV topotecan. The objectives of Part 1b are to describe the 1) progression free survival rate at 12 weeks, 2) objective response rate (ORR), 3) progression free survival (PFS), 4) overall survival (OS), and 5) safety profile.

Part 2: a randomized, efficacy study of the nal-IRI (N=210) versus topotecan (N=210). The primary objective of Part 2 is to compare overall survival following treatment with irinotecan liposome injection with overall survival following treatment with IV topotecan.

The secondary objectives of Part 2 are to compare the following between the treatment arms: 1) progression free survival (PFS), 2) objective response rate (ORR), 3) proportion of patients with symptom improvement in cough, in dyspnea and in fatigue as measured by the European Organization for Research and Treatment of Cancer Quality of Life Questionnaire (EORTC QLQ-C30) and Lung Cancer 13 (LC13) and 4) safety profile.

Exploratory Objectives (Part 1 and Part 2) include: 1) To describe QTcF following treatment with irinotecan liposome injection (Part 1 only), 2) To explore the biomarkers associated with efficacy and safety following treatment with irinotecan liposome injection, 3) To describe the association between UGT1A1 genotype, SN-38 concentration (irinotecan liposome injection treated patients only) and safety, 4) To evaluate the relationship between plasma pharmacokinetics of irinotecan liposome injection and efficacy and safety in this patient population, 5) To compare the rate of development/time to development of CNS progression and development of new CNS metastases, 6) To compare time to treatment failure (TTF) between treatment arms and 7) To compare patient-reported outcomes (PROs) between treatment arms using EORTC-QLQ-C30, EORTC-QLQ-LC13 and EQ-5D-5L.

Both Part 1 and Part 2 will consist of three phases: a screening phase, a Treatment/Active follow-up phase and a long term follow-up phase. The Treatment/Active follow-up phase is the period for the first dose of the study drug through the decision to permanently discontinue study drug treatment. The Long Term follow-up phase is a monthly follow-up for overall survival.

Part 1a

The initial number of patients to be enrolled in the Part 1a safety run-in is 6 patients evaluable for safety. This initial cohort of patients will be treated with irinotecan liposome injection 70 mg/m² every 2 weeks. Dose limiting toxicities (DLTs) will be evaluated during the first 28 days of treatment (or 14 days after the 2nd dose of study treatment if there is a treatment delay) to determine if the dose is tolerable. If 2 or more patients receiving irinotecan liposome injection 70 mg/m² every 2 weeks have DLTs then the dose will be declared not tolerable. In all other cases an additional cohort of 6 patients treated with irinotecan liposome injection starting at 90 mg/m² will be enrolled. The 90 mg/m² cohort will only be enrolled if the overall experience in the initial 6 patients treated in the 70 mg/m² cohort is judged to be safe enough to reasonably expect that the 90 mg/m² dose will be tolerable in the assessment of the Part 1 investigators and the Sponsor. Evaluation of DLTs will follow the same guidelines as the first cohort. If 2 or more patients have DLTs at the 90 mg/m² dose then that dose will be considered to exceed the optimal safety and tolerability criteria, and 70 mg/m² will be designated as the dose for Part 1b and Part 1b will initiate administering 70 mg/m² of irinotecan liposome injection. If there is 0 or 1 DLT within the safety evaluation period with the 90 mg/m² dose, then the decision of which dose to use for Part 1b will be made by Part 1 investigators and the Sponsor based on the entire safety experience of both cohorts.
All patients who received study drug will be evaluable for DLT and safety. The following adverse events should be considered as DLTs if they occur during the first 28 days of treatment (or 14 days after the 2nd dose of study treatment if there is a treatment delay according to section 6.2) and are deemed related to the study treatment by the investigator: Grade 4 neutropenia or thrombocytopenia that does not resolve within 7 days and grade 4 anemia of any duration
Inability to begin subsequent treatment course within 14 days of the scheduled date, due to drug-related toxicity
Grade 3-4 neutropenia complicated by fever ≥38.5° C. (i.e. febrile neutropenia) and/or by infection
Any grade 4 non-hematologic toxicity with the exception of the following:
   Fatigue/asthenia <2 weeks
   Nausea and vomiting lasting ≤3 days duration (only considered dose limiting if they last >72 hours after treatment with an optimal anti-emetic treatment)
   Diarrhea ≤3 days duration (only considered dose limiting if diarrhea lasts >72 hours after treatment with an optimal anti-diarrheal regimen)
Grade 3 non-hematologic toxicity with the exception of the following:
   Any gastrointestinal disorder and dehydration (with associated signs and symptoms) unless grade 3 toxicity persists despite optimal medical management for >72 hours,
   Pain unless grade 3 toxicity persists despite optimal medical management,
   Fatigue, fever, flu like symptoms, infections and infestations
   Infusion reaction (and associated symptoms) unless it occurs following steroid premedication
   Hepatic and kidney function abnormalities, and electrolyte abnormalities if they persist, despite optimal medical management

The determination whether an adverse event is considered a DLT will be made following discussion between the investigators and the Sponsor and confirmed by the safety review committee (i.e. the Part 1a Investigators and the Medical Monitor(s) of the Sponsor). Other adverse events that are deemed related to study treatment can also be considered a DLT event at the discretion of the safety review committee. Safety review meetings between investigators and sponsor will occur regularly during Part 1a of the study with at least monthly meetings, or more frequently, if required.

Part 1b

Following the determination of the nal-IRI dose in Part 1a, Part 1b of the study will be initiated. In Part 1b, approximately 50 eligible patients will be randomized in a 1:1 ratio between the experimental arm (Arm 1a: 90 mg/m² of nal-IRI, every 2 weeks), and the control arm (Arm 1b: topotecan 1.5 mg/m2 IV for 5 days, every 21 days). Patients will be randomized to the treatment arms using an Interactive Web Response System (IWRS) at a central location. In order to reduce imbalance with regard to prognostic factors used for stratification in the randomization for Part 2, randomization in Part 1b will use a minimization procedure accounting for the Part 2 stratification factors.

Platinum resistant patients are defined as patients with disease that either progressed during first-line platinum containing therapy or within 90 days of its completion. Platinum sensitive patients are defined as patients with disease that progressed after 90 days of completion of first line platinum containing therapy. To retain a distribution of platinum sensitivity to first-line treatment groups in accordance with previously published studies (von Pawel, 2014), no more than 30 patients will be randomized from either platinum sensitive or platinum resistant patients in Part 1b.

Safety and efficacy results from Part 1b will determine if the study proceeds (or not) to Part 2. The study will be stopped if both of the following stopping criteria are met:

PFS (based on investigator assessment) rate at 12 weeks for irinotecan liposome injection is less than 50% AND PFS (based on investigator assessment) rate at 12 weeks for IV topotecan exceeds that of irinotecan liposome injection by at least 5 percentage points

If the stopping criteria are not met, the final decision to proceed to Part 2 will be made by the Sponsor in consultation with the academic steering committee of the study after consideration of all available efficacy and safety data from Part 1 of the study.

Part 2:

If the stopping criteria from Part 1b are not met and the decision is made to proceed to Part 2 of the study, approximately 420 eligible patients will be randomized in a 1:1 ratio between the experimental arm (Arm 2a: 90 mg/m² of irinotecan liposome injection), and the control arm (Arm 2b: IV topotecan). Patients will be randomized to the treatment arms using an Interactive Web Response System (IWRS) at a central location. Randomization will be stratified, based on the following factors:
Disease stage (limited vs extensive) at diagnosis
Region (North America vs Asia vs Other)
Platinum sensitivity (sensitive vs resistant)
Performance status (ECOG 0 vs. 1)
Prior immunotherapy (yes vs. no)

Only region and platinum sensitive vs. resistant will be used for the efficacy analysis.

Tumor responses will be measured and recorded, every 6 weeks (+/−1 week) by using the RECIST guidelines (version 1.1). The tumor assessment at baseline is CT with contrast (chest/abdomen required and pelvis if clinically indicated) and brain Mill with contrast (CT of brain is acceptable). Each follow-up tumor assessment should use the same assessment as performed at baseline, unless medically contraindicated. All patients will have imaging of the brain at baseline and at each assessment. Patients who discontinue study treatment, for reasons other than objective disease progression, should continue to be followed-up until radiological documentation of progressive disease. The Sponsor will collect and store all tumor measurement images on all patients throughout the study; however, local radiologist and/or PI assessment will determine disease progression. A review of the scans may be performed by the Sponsor for an independent analysis, including analysis of PFS and/or ORR. All patients will be followed at least monthly until death or study closure, whichever occurs first.

A quality of life assessment will be performed using the EORTC-QLQ-C30, EORTC-QLQ-LC13, and the EuroQoL five-dimension, five level health status questionnaire (EQ-5D-5L) in Part 1b and Part 2 only. Both instruments will be administered before randomization and prior to dosing at 6 week intervals following start of treatment and at treatment discontinuation and at the 30-day follow-up visit.

Adverse events (AEs) will be evaluated according to the National Cancer Institute's Common Terminology Criteria for Adverse Events version 4.03 (CTCAE v4.03). For summary of AEs, events will be coded using the latest MedDRA dictionary version.

The primary analysis is planned when at least 333 OS events have occurred. An interim analysis for futility is planned to occur at 30% information time, after at least 100 OS events have occurred. In the event that the trial continues, an interim analysis will be conducted when at least 210 OS events (63% information time, at 50% of anticipated death events) have occurred to assess the potential for early stopping due to efficacy of the experimental treatment regimen.

A regular review of safety data will be conducted for Part 2 by an independent Data Monitoring Committee (DMC). The DMC will consist of oncology and statistical experts independent of the Sponsor. The first safety review of the DMC will take place in Part 2 after the 30th patient is treated for at least one cycle or after the 30th patient discontinued study drug, whichever occurs first. The timing and details of subsequent data reviews will be detailed in the DMC charter. Items reviewed on a regular basis will include (but not limited to) safety events, results of PK testing, and UGT1A1*28 genotype from central testing with particular attention to determine whether any study procedure needs to be modified for patients who are homozygous for UGT1A1*28.

Pharmacokinetics

Plasma samples for PK will be collected in Cycle 1 only at the following time points:

Part 1a, and Part 1b, Arm 1a (nal-IRI arm; cycle 1 only):
Day 1: Pre-dose
Day 1: End of nal-IRI infusion
Day 2: Approximately 24 hours after end of infusion
Day 8: Cycle 1, Day 8 (+/−1 day), at any time of day
Day 15: Pre-dose
Day 15: End of nal-IRI infusion

Part 1b, Arm 1b (topotecan arm; cycle 1 only):
Day 1: Pre-dose
Day 1: End of topotecan infusion
Day 1, 2 or 3: Two additional samples between 1.5 and 4 hours after the start of infusion. Each sample must be collected at least 1 hour apart. It is preferred to collect these samples on day 1; however these two additional samples can be collected on day 2 or day 3.

Part 2, Arm 2a (irinotecan liposome injection arm; cycle 1 only):
Day 1: Pre-dose
Day 1: End of irinotecan liposome injection infusion
Day 1: Between 2.5 and 6 hours after the start of infusion
Day 2-6 (Optional): anytime between 1 and 5 days after the start of infusion
Day 8: Cycle 1 Day 8 (+/−1 day), at any time of day.

Study Population

Inclusion Criteria

Disease Specific Inclusion Criteria

1) histopathologically or cytologically confirmed small cell lung cancer according to the International Association for the Study of Lung Cancer (IASLC) histopathological classification. Mixed or combined subtypes according to the IASLC are not allowed; 2) Evaluable disease as defined by RECIST v1.1 guidelines (patients with non-target lesions only are eligible) 3) Progression on or after first-line platinum based chemotherapy (carboplatin or cisplatin) or chemo-radiation including platinum-based chemotherapy for treatment of limited or extensive stage SCLC; and 4) Recovered from the effects of any prior chemotherapy, surgery, radiotherapy or other anti-neoplastic therapy (recovered to grade 1 or better, with the exception of alopecia).

Hematologic, Biochemical and Organ Function Inclusion Criteria:

Adequate bone marrow reserves as evidenced by:
ANC>1,500 cells/μ1 without the use of hematopoietic growth factors; and
Platelet count>100,000 cells/μ1; and
Hemoglobin>9 g/dL; transfusions are allowed

Adequate hepatic function as evidenced by:
Serum total bilirubin within normal range for the institution
Aspartate aminotransferase (AST) and alanine aminotransferase (ALT)≤2.5×ULN (≤5×ULN is acceptable if liver metastases are present)

Adequate renal function as evidenced by a serum creatinine ≤1.5×ULN and creatinine clearance ≥40 mL/min. Actual body weight should be used for calculating creatinine clearance using the Cockcroft-Gault Equation (except for patients with body mass index (BMI)>30 kg/m2 when lean body weight should be used instead):

$$Serum Creatinine ( mg / min ) \begin{matrix}( 140 - Age \underline{( years ) ) \times ( Weight ( kg ) )} \\ 72 \times Serum Creatinine ( mg / \underline{dL )}\end{matrix} \underline{\times Sex}$$

Where Sex=1 for males and 0.85 for females.

ECG without any clinically significant findings

Recovered from the effects of any prior chemotherapy, surgery, radiotherapy or other anti-neoplastic therapy

Required to participate in the translational research component of the trial, unless prohibited by local regulations, and provide archived tumor tissue (if available)

At least 18 years of age

Able to understand and sign an informed consent (or have a legal representative who is able to do so)

Patients must meet all the inclusion criteria listed above and none of the following exclusion criteria:

General Exclusion Criteria

1) Any medical or social condition deemed by the Investigator to be likely to interfere with a patient's ability to sign informed consent, cooperate and participate in the study, or interfere with the interpretation of the results;

2) Pregnant or breast feeding; females of child-bearing potential must test negative for pregnancy at the time of enrollment based on a urine or serum pregnancy test. Both male and female patients of reproductive potential must agree to use a highly effective method of birth control, during the study and for 4 months following the last dose of study drug.

Disease Specific Exclusion Criteria

1) Prior treatment regimens with irinotecan, topotecan or any other topoisomerase I inhibitor including investigational topoisomerase I inhibitors;

2) Patients with large cell neuroendocrine carcinoma;

3) Patients who have had more than one regimen of prior cytotoxic chemotherapy

4) More than one line of immunotherapy (e.g. nivolumab, pembrolizumab, ipilimumab, atezolizumab, tremelimumab and/or durvalumab). One line of immunotherapy is defined as the following: monotherapy or combination of immunotherapy agents given as either (i) in combination with chemotherapy followed by immunotherapy maintenance in the first line setting, (ii) only as a maintenance following response to first-line chemotherapy or (iii) immunotherapy given as second line treatment following progression;

5) Patients with a history of immunotherapy induced colitis;

6) Any prior systemic treatment other than 1 line of platinum-containing regimen or immunotherapy as described above;

7) Patients with the following CNS metastasis:

i) Patients who have developed new or progressive brain metastasis following prophylactic and/or therapeutic cranial radiation (whole brain stereotactic radiation).

ii) Patients with symptomatic CNS metastasis (a patient with brain metastasis who received cranial radiotherapy is eligible if asymptomatic for neurological symptoms for ≥2 weeks after cranial radiotherapy and is off corticosteroids for treatment of CNS metastasis. Patients with asymptomatic brain metastases are eligible to be enrolled directly to the study).

iii) Patients with carcinomatous meningitis;

8) Unable to discontinue the use of strong CYP3A4 or UGT1A1 inhibitors at least 1 week or strong CYP3A4 inducers at least 2 weeks prior to receiving the first dose of irinotecan liposome injection;

9) Presence of another active malignancy; or

10) Investigational therapy administered within 4 weeks, or within a time interval less than at least 5 half-lives of the investigational agent, whichever is less, prior to the first scheduled day of dosing in this study.

Hematologic, Biochemical and Organ Function Exclusion Criteria

1) Severe arterial thromboembolic events (e.g. myocardial infarction, unstable angina pectoris, stroke) less than 6 months before inclusion; 2) NYHA Class III or IV congestive heart failure, ventricular arrhythmias, or uncontrolled blood pressure; 3) Active infection (e.g. acute bacterial infection, tuberculosis, active hepatitis B or active HIV) which in the investigator's opinion might compromise the patient's participation in the trial or affect the study outcome; 4) Known hypersensitivity to any of the components of irinotecan liposome injection, other liposomal products, or topotecan; or Clinically significant gastrointestinal disorder including hepatic disorders, bleeding, inflammation, occlusion, or diarrhea>grade 1.

Length of Study

It is intended that patients will be treated until disease progression or unacceptable toxicity. Following treatment discontinuation, patients will return to the study site for a 30 day follow up visit. After this visit, patients will continue to be followed for overall survival status by phone or a visit to the study site once every month until death or study closure, whichever occurs first.

Method of Assigning Patients to Treatment Groups

Part 1a:

After all screening assessments have been completed and the first patient reported outcome assessment has been completed, eligible patients will enter Part 1a.

Part 1b:

Part 1b will be initiated following dose selection in Part 1a.

After all screening assessments have been completed and the first patient reported outcome assessment has been completed, eligible patients will be randomized using a computerized interactive web response system (IWRS), in a 1:1 ratio, to one of the following treatment arms: Randomization in Part 1b will use a minimization procedure (McEntegart, 2003) accounting for the Part 2 stratification factors.

Arm 1a (experimental arm): irinotecan liposome injection

Arm 1b (control arm): IV topotecan

Randomization must occur within 7 days of planned dosing.

Part 2:

Part 2 will be initiated upon passing the stopping criteria and based on the decision of the Sponsor in consultation with the academic steering committee.

After all screening assessments have been completed and the first patient reported outcome assessment has been completed, eligible patients will be randomized using a computerized interactive web response system (IWRS), in a 1:1 ratio, to one of the following treatment arms:

Arm 2a (experimental arm): irinotecan liposome injection

Arm 2b (control arm): IV topotecan

Randomization must occur within 7 days of planned dosing. The randomization will be stratified based on the following prognostic factors:
Region (North America vs. Asia vs. Other)
Platinum sensitivity (sensitive vs. resistant)
Disease stage (limited vs. extensive) at diagnosis
Performance status (ECOG 0 vs. 1)
Prior immunotherapy (yes vs. no)

Platinum resistant patients are defined as patients with disease that either progressed during first-line platinum containing therapy or within 90 days of its completion. Platinum sensitive patients are defined as patients with disease that progressed after 90 days of completion of first line platinum containing therapy.

Administration of Irinotecan Liposome Injection

Part 1a:

Irinotecan liposome injection will be administered at a dose of 70 mg/m2 (strength expressed based on irinotecan free base; approximately equivalent to 80 mg/m2 of the anhydrous salt) IV over 90 minutes, every 2 weeks in a 6-week cycle. Should the 70 mg/m2 dose be deemed tolerable and 90 mg/m2 is explored, irinotecan liposome injection should be administered at 90 mg/m2 (strength expressed based on irinotecan free base; approximately equivalent to 100 mg/m2 of the anhydrous salt) IV over 90 minutes, every 2 weeks in a 6-week cycle.

Part 1b & 2:

Irinotecan liposome injection will be administered at a dose of 90 mg/m2 (strength expressed based on irinotecan free base; approximately equivalent to 100 mg/m2 of the anhydrous salt): IV over 90 minutes, every 2 weeks in a 6-week cycle (unless deemed unacceptable in Part 1).

Prior to administration, the appropriate dose of irinotecan liposome injection must be diluted in 5% Dextrose Injection (D5W) or 0.9% Sodium Chloride Injection to a final volume of 500 mL. Care should be taken not to use any diluents other than D5W or 0.9% sodium chloride.

UGT1A1*28 Monitoring

UGT1A1*28 genotype will be collected on all patients and assessed centrally. Results will be provided to the site and to the Sponsor. Sites will also be asked to include the result from the UGT1A1*28 genotyping on the SAE reporting form.

All patients treated with irinotecan liposome injection, regardless of the results of the UGT1A1*28 genotype, will be treated with the same starting dose of irinotecan liposome injection and will follow the same dose reduction rules. During the regular safety monitoring of patients during the study, as will be conducted by the sponsor medical monitor(s) and by the DMC (in Part 2), the safety and PK of UGT1A1*28 homozygous patients will be compared to those who are non-homozygous for UGT1A1*28 to determine whether any different dosing strategy (such as a lower starting dose and/or different dose reduction for irinotecan liposome injection) is required for patients who are homozygous for UGT1A1*28. The first safety DMC meeting will occur after the 30th patient completed once cycle of treatment or discontinued treatment, whichever occurs first. No association between UGT1A1*28 and safety is expected in patients treated with topotecan.

Study Treatments

Irinotecan Liposome Injection:

Part 1a: (Safety Run-In)

Irinotecan liposome injection 70 mg/m² (strength expressed as irinotecan free base; approximately equivalent to 80 mg/m² of the anhydrous salt) IV over 90 minutes, every 2 weeks in a 6 week cycle) OR irinotecan liposome injection 90 mg/m² (strength expressed as irinotecan free base; approximately equivalent to 100 mg/m² of the anhydrous salt) IV over 90 minutes, every 2 weeks in a 6 week cycle.

Part 1b and Part 2:

Arm 1a and 2a (Experimental Arm):

Irinotecan liposome injection 90 mg/m² (strength expressed as irinotecan free base; approximately equivalent to 100 mg/m² of the anhydrous salt): IV over 90 minutes, every 2 weeks in a 6 week cycle (unless deemed unacceptable in Part 1).

Arm 1b and 2b (Control Arm):

Topotecan 1.5 mg/m²: IV over 30 minutes daily for 5 consecutive days, every 3 weeks in a 6 week cycle.

Irinotecan Liposome Injection:

Part 1a, Part 1b Arm 1a and Part 2 Arm 2a:

Supportive care measures should follow the guidelines outlined in the prescribing information for ONIVYDE®. Up to two dose reductions of irinotecan liposome injection are permitted for toxicities. The use of prophylactic G-CSF (both short and long acting growth factor is acceptable, based on investigator preference) with the second or later doses of irinotecan liposome injection is allowed, based on investigator judgment.

Topotecan:

Part 1b Arm 1b and Part 2 Arm 2b (IV Topotecan)

The intended dose for topotecan is 1.5 mg/m2 IV for 5 consecutive days every 3 weeks. The dose, administration and dose reductions should follow the guidance as outlined in the prescribing information for IV topotecan.

Patients randomized to treatment with topotecan should be considered for prophylactic G-CSF in all cycles starting 24 hours following the last dose (both short and long acting growth factor is acceptable, based on investigator preference). Up to two dose reductions of topotecan per patient are permitted for toxicities. Dose delays are permitted to allow recovery from treatment-associated toxicities. Prophylactic antibiotics are recommended for patients at high risk of infectious complications.

Investigational Product:

Irinotecan liposome injection (also known as nal-IRI, pegylated liposomal irinotecan hydrochloride trihydrate, MM-398, PEP02, BAX2398 and ONIVYDE®) is a sterile, white to slightly yellow opaque isotonic liposomal dispersion. Each 10 mL single-dose vial contains 43 mg irinotecan free base at a concentration of 4.3 mg/mL. The liposome is a unilamellar lipid bilayer vesicle, approximately 110 nm in diameter, which encapsulates an aqueous space containing irinotecan in a gelated or precipitated state as the sucrose octasulfate salt. It will be supplied as sterile, single-use vials containing 43 mg irinotecan free base at a concentration of 4.3 mg/mL. Irinotecan liposome injection must be stored refrigerated (2 to 8° C., 36 to 46° F.) with protection from light. Do not freeze.

Part 1a

A dose will be decided to be acceptable for proceeding to Part 1b if the number of patients with DLTs does not exceed 1 in a cohort of 6 patients. Based on this rule, the probabilities to proceed to Part 1b at a dose as a function of true DLT probability rate are shown in Table 6.

TABLE 6

| True rate of unacceptable | Probability to advance |
|---|---|
| Toxicity | to randomization |
| 0.05 | 0.97 |
| 0.10 | 0.89 |
| 0.15 | 0.77 |
| 0.20 | 0.65 |
| 0.25 | 0.53 |
| 0.30 | 0.42 |
| 0.35 | 0.32 |
| 0.40 | 0.23 |

Part 1b

The purpose of Part 1b is to provide a pilot sample of safety and efficacy data in a randomized setting. The sample size for Part 1b was selected for practical purposes to enable curtailment of the study if irinotecan liposome injection is observed to be substantially inferior to topotecan with regard to benefit/risk.

An efficacy rule, based on the observed PFS rate at 12 weeks, is implemented in this protocol as a formal stopping rule, while additional data will also be considered and may also result in a decision to not proceed to Part 2. The operating characteristics of the formal stopping rule, given the study design in Part 1b, are described below.

Using a binomial distribution to approximate and assuming the true proportion of progression-free patients at 12 weeks within the control group is 0.55, the probability that the study would be stopped, as a function of the true rate for the irinotecan liposome injection arm, is shown in Table 7.

TABLE 7

| Irinotecan liposome injection | Absolute Δ | Probability stop |
|---|---|---|
| PFS rate at 12 weeks | to control | given rules |
| 0.75 | 0.20 | 0.002 |
| 0.70 | 0.15 | 0.011 |
| 0.65 | 0.10 | 0.038 |
| 0.60 | 0.05 | 0.101 |
| 0.55 | 0 | 0.211 |
| 0.50 | −0.05 | 0.363 |
| 0.45 | −0.10 | 0.536 |
| 0.40 | −0.15 | 0.698 |
| 0.35 | −0.20 | 0.827 |

A final treatment comparison of PFS will be carried out via a log-rank test when tumor assessments have been completed for all patients in Part 1b. If the censoring rate is assumed to be 10%, it is expected that 45 events would have occurred at the time of the final analysis. If the PFS hazard ratio is 0.64 (e.g. irinotecan liposome injection extends median PFS from 3.5 to 5.5 months), then this analysis would have approximate 75% power to detect the treatment difference with a one-sided level 0.20 test.

Part 2

The primary endpoint is overall survival (OS).

A total of 420 patients will be randomized in a 1:1 ratio to the two treatment arms. Follow-up until at least 333 OS events are observed across the two treatment arms provides at least 85% power to detect a true hazard ratio of HR≤0.714 (mOS: 7.5 v 10.5 months) using a stratified log-rank test (stratified by region (North America vs. Asia vs. Other) and platinum sensitivity (sensitive vs. resistant)) with overall 1-sided significance level of 0.025 (adjusted for interim analyses).

Assuming enrollment over 25 months with a ramp-up to 21 patients per month and lost-to-follow-up rate of 5% across both treatment arms, the timing of the primary analysis is expected to be at 39 months.

An interim analysis for futility will be conducted when approximately 30% of the planned final number of OS events (i.e., 100 of 333 OS events) has been observed in the intent-to-treat (ITT) population. In the event that the study proceeds, a second interim analysis will occur to evaluate both futility and efficacy when approximately 210 OS events (63% of planned OS events and 50% of expected events in the entire study population) have occurred.

General:

Categorical variables will be summarized by frequency distributions (number and percentages of patients) and continuous variables will be summarized by descriptive statistics (mean, standard deviation, median, minimum, maximum).

The efficacy and safety of nal-IRI in Part 1 will be reported descriptively using the same outcome measures as in Part 2. In addition, adverse events occurring in Part 1 of the study will be described in detail.

Patients enrolled and treated with study drug in Part 1 will comprise the Part 1 safety population. The safety and efficacy of these patients will be presented descriptively.

Patients randomized in Part 2 will comprise the intent-to-treat (ITT) population. This will be the population that is evaluated in comparison to evaluate the efficacy of the experimental arm. In the ITT analyses of efficacy, each patient will be considered according to the randomized treatment assignment. Patients who received any part of any study drug will define the Part 2 safety population.

For stratified analyses, stratification factors will be the randomization stratification factors of region (North America, Asia, Other) and platinum sensitivity (sensitive, resistant). Classification of stratification factors will be according to the randomization.

Primary Efficacy Analysis (Part 2):

OS is defined as the number of months from the date of randomization to the date of death. Patients without observed death at the time of the primary analysis will have OS censored according to the last recorded date alive.

The primary analysis will be performed using a stratified log-rank test comparing the OS difference between two treatment arms with 1-sided level of significance at 0.025. Stratification factors will include the randomization stratification factors and classification will be according to the randomization. Kaplan-Meier methods will be used to estimate median OS (with 95% confidence intervals) and to display OS time graphically. A stratified Cox proportional hazards model will be used to estimate hazard ratio and its corresponding 95% confidence interval. Sensitivity analyses for OS will be described in the Statistical Analysis Plan (SAP).

Key Secondary Analyses (Part 2):

Key secondary endpoints are PFS, ORR, proportion of patients with symptom improvement in dyspnea, in cough, and in fatigue.

Key secondary endpoints will tested no more than once. If the primary endpoint of OS is statistically significant at the interim, testing of secondary endpoints will be tested at the interim. Otherwise secondary endpoints will be tested at the final OS analysis if OS if found to be statistically significant at that analysis. Hypothesis testing of key secondary endpoints will be conducted in a stage wise hierarchical manner (Glimm, E, et al., Statistics in Medicine 2010 29:219-228).

The nominal level for comparison of PFS will depend on whether the test is performed at the interim or at the planned final analysis and will incorporate an α-spending function similar to that used for OS. If OS and PFS are both significant, then ORR and EORTC-QLQ symptoms will be tested at 1-sided 0.025 level (nominal α adjusted based on spending function, as described for PFS) with each p-value adjusted using the Benjamini-Hochberg correction (Benjamini & Hochberg, J. Royal Statistical Soc. B 2005 57, 289-300) for one-sided a level testing of 4 planned comparisons. Adjusted p-values will be reported, using SAS PROC MULTTEST with FDR option or equivalent algorithm. Any parameter which is not statistically significant will be regarded as descriptive and exploratory.

Progression-Free Survival:

Progression-free survival is the time from randomization to the first documented objective disease progression (PD) using RECIST v1.1 or death due to any cause, whichever occurs first. Determination of PFS will be per investigator assessment. If neither death nor progression is observed, data will be censored on the date of the last observed tumor assessment date. Patients without a valid tumor response evaluation at randomization will be censored on the date of randomization. Patients starting a new anti-cancer treatment prior to documented PD will be censored at the date of the last observed tumor assessment prior to start of the new treatment. Patients with documented PD or death after an unacceptable long interval (i.e., 2 or more missed or indeterminate scheduled assessments) will be censored at the time of the last observed non-PD tumor assessment date prior to progression or death.

The difference in PFS between treatments will be evaluated using a stratified log-rank test. Kaplan-Meier methods will be used to estimate median PFS (with 95% confidence intervals) and to display PFS time graphically. A stratified Cox proportional hazards model will be used to estimate the PFS hazard ratio and its corresponding 95% confidence interval.

The difference in PFS between treatments will be evaluated using a stratified log-rank test (stratified by region and platinum sensitivity). Kaplan-Meier methods will be used to estimate median PFS (with 95% confidence intervals) and to display PFS time graphically. A stratified Cox proportional hazards model will be used to estimate the PFS hazard ratio and its corresponding 95% confidence interval. Sensitivity analyses for PFS will be described in the SAP.

Objective Response:

Objective response rate (ORR) is the proportion of patients who achieve partial response or complete response according to RECIST v1.1 guidelines. An estimate of the ORR and its 95% CI will be calculated. The difference in ORR between treatment groups will be compared using Cochran-Mantel-Haenszel method, stratified by region and platinum sensitivity.

Proportion of Patients with Improvement of Lung Cancer Symptoms:

This secondary analysis will consider the patient-reported EORTC-QLQ-LC13 symptom scales for cough, dyspnea, and fatigue, as these are considered most clearly to be disease-related and evaluable for treatment impact with regard to the proportion of patients with improvement. The remaining EORTC-QLQ symptom domains will be assessed in exploratory analyses.

Symptom improvement is defined as achievement and 6-week maintenance of symptom subscale scores at least 10 percentage points of scale (following transformation to 0-100 scale) below baseline. Response classifications will be tabulated by treatment group and statistical analyses will compare the proportions of responders for a given symptom.

For each symptom, the proportion of patients with improvement will be tabulated by treatment group with 95% confidence intervals based on a Normal approximation. The difference in the proportion of patients with symptom improvement will be presented with corresponding 95% confidence intervals. The proportion of patients with improvement in a symptom will be compared between treatment regimens using Cochran-Mantel-Haenszel method, stratified by region and platinum sensitivity.

Safety Analysis:

Safety analyses (adverse events and laboratory analyses) will be performed using the safety population, defined as all patients receiving any study drug. Treatment assignment will be according to actual treatment received. Adverse events will be coded using the latest MedDRA dictionary. Severity will be graded according to the NCI CTCAE version 4.03.

Treatment-emergent adverse events (TEAEs) are defined as any adverse events reported from the date of first study drug exposure to 30 days after the last date of study drug exposure. Frequency and percentages of patients will be summarized for: any grade TEAE, grade 3 or higher TEAE, study-drug related TEAE, serious TEAE, TEAE leading to dose modification, and TEAE leading to study drug discontinuation. Adverse events will be summarized by System Organ Class and preferred term. All adverse event data will be listed by patient.

Laboratory data will be summarized according to parameter type. Where applicable, toxicity grading for laboratory safety parameters will be assigned based on NCI CTCAE version 4.03 criteria.

QTcF Analyses:

The potential of QTcF prolongation with irinotecan liposome injection treatment will be evaluated in patients receiving irinotecan liposome injection in Part 1 of this study. For the primary QTcF prolongation analysis, the predicted changes in QTcF will be obtained from the exposure-QTcF relationship using mixed-effect modeling. Sensitivity analyses will be conducted by evaluating by time point and categorical analyses.

EORTC-QLQ Outcomes

Analysis of the EORTC-QLQ-C30 questionnaires will be performed in accordance with the EORTC guidelines (Fayers, 2001). The subscales of the EORTC QLQ-C30 and the QLQ-LC13 will be scored based on the EORTC scoring manual. Scores will be standardized such that higher scores on the EORTC QLQ-C30 or the QLQ-LC13 will represent higher (“better”) levels of functioning and/or a higher (“worse”) level of symptoms.

Analysis methods for the proportion of patients with symptom improvement are as described in Key Secondary Analysis (section 11.5.2.3).

Frequency tables by treatment group will be reported for the proportion of patients with symptom improvement for each QLQ-C30 and QLQ-LC13 subscale. Details of additional EORTC-QLQ analyses will be provided in the Statistical Analysis Plan.

Raw standardized subscale scores and changes from baseline in over time will be reported. Mean change scores will be compared between treatment groups descriptively and may be explored via longitudinal modeling (i.e., covariate analysis and repeated measures modeling)

EQ-5D-5L:

Raw score and change from baseline in over time will be reported. Mean change scores will be compared between treatment groups descriptively and explored via longitudinal modeling (i.e., covariate analysis and repeated measures modeling).

Time to CNS Progression:

Is defined as time from randomization to development of CNS progression as defined by the RANO-BM working group (Lin et al Lancet Oncology 2015). Time to CNS progression will be described by Kaplan-Meier methods and treatments will be compared using stratified log-rank test.

Pharmacokinetics (PK) and Pharmacodynamics (PD) Analysis:

Plasma pharmacokinetics (PK) of total irinotecan, SN-38, and topotecan will be quantified from the concentration samples using nonlinear mixed effect modeling. The initial PK analysis will use the empirical Bayesian estimation, however, additional covariate analyses will be performed to evaluate alternative baseline factors specific to SCLC. The resulting PK estimates will be used to evaluate the association between PK and PD (efficacy and safety endpoints). Topotecan PK will be used to provide additional data to understand the results from Part 1b, by comparing the distribution and the association of PK to efficacy/safety in this study to historical values.

Dose Modifications

All dose modifications should be based on worst preceding toxicity.

TABLE 8
Recommended Dose Modifications for Irinotecan Liposome Injection

| | | Starting Dose | |
|---|---|---|---|
| Toxicity NCI CTCAE v4.03 Grade^{b} Occurrence | | 70 mg/m² | 90 mg/m² |
| Neutropenia, leukopenia, or | First occurrence | 50 mg/m² | 70 mg/m² |
| thrombocytopenia Grade 3 or 4 | Second occurrence | 43 mg/m² | 50 mg/m² |

| | | |
|---|---|---|
| Neutropenic fever | Third occurrence | Discontinue treatment |

A new cycle of therapy should not begin until the absolute neutrophil count
is ≥1500/mm³ (1.5 × 10⁹/L)
A new cycle of therapy should not begin until the platelet count is ≥100,000/mm³
(100 × 109/L)
Nonhematological toxicities:

| All nonhematological toxicities | Withhold ONIVYDE. Initiate loperamide for late | | |
|---|---|---|---|
| (except asthenia and anorexia): | onset diarrhea of any severity. Administer | | |
| Grade 3 or 4 | intravenous or subcutaneous atropine 0.25 to 1 mg | | |
| | (unless clinically contraindicated) for early onset | | |
| | diarrhea of any severity. Upon recovery to < Grade | | |
| | 1, resume ONIVYDE as below: | | |
| | First occurrence | 50 mg/m² | 70 mg/m² |
| | Second occurrence | 43 mg/m² | 50 mg/m² |

| | |
|---|---|
| Third occurrence | Discontinue treatment |

A new cycle of therapy should not begin until serum chemistry parameters resolve
to ≤ Grade 1
A new cycle of therapy should not begin until nonhematological toxicities resolve
to ≤ Grade 1
For Grade ≥ 3 nausea and vomiting, reduce dose only if occur despite optimal
anti-emetic therapy
Asthenia and Grade 3 anorexia do not require any dose modifications

| | | |
|---|---|---|
| Interstitial lung disease | First occurrence | Discontinue treatment |
| Severe hypersensitivity reaction | First occurrence | Discontinue treatment |

^{a} All doses mentioned are based on irinotecan free base
^{b}National Cancer Institute Common Terminology Criteria for Adverse Events, version 4.03

Topotecan for Injection

Topotecan should only be started in patients with a baseline neutrophil count of greater than or equal to 1,500/mm3 (1.5×109/L) and a platelet count greater than or equal to 100,000/mm³ (100×10⁹/L).

Topotecan should not be administered in subsequent cycles unless the neutrophil count is ≥1×10⁹/l, the platelet count is ≥100×10⁹/l, and the hemoglobin level is ≥9 g/dl (after transfusion if necessary). Treatment should be delayed to allow sufficient time for recovery and upon recovery, treatment should be administered according to the guidelines in Table 9 below.

Dose reduction of topotecan should occur in case of the following toxicities:
Grade 4 neutropenia (ANC <500/mm3 or <0.5×109/L);
Grade 4 thrombocytopenia (platelet count <25,000/mm3 or <0.5×109/L)
Grade 3 or 4 non-hematological toxicity except nausea and vomiting. In case of nausea and vomiting, dose reduction should occur if Grade 3 or 4 toxicity occurs despite optimal medical management

Dose reduction decisions should be based on worst preceding toxicity. Moving from dose level 0 to dose level 2 is permitted. Prophylactic antibiotics are recommended for patients at high risk of infectious complications.

Up to two dose reductions of topotecan per patient are permitted for toxicities as shown in Table 9. If a third dose reduction is needed to manage a toxicity, topotecan treatment should be discontinued.

TABLE 9
Recommended topotecan Dose Modification
Scheme for Subsequent Cycles

| Dose Level | Dose Modifications |
|---|---|
| 0 | 1.5 mg/m² IV days 1-5 |
| −1 | 1.25 mg/m² IV days 1-5 |
| −2 | 1.0 mg/m² IV days 1-5 |

The dose of topotecan in patients should be reduced to 0.75 mg/m2/day for five consecutive days if the creatinine clearance is between 20 and 39 mL/min.

Topotecan should be discontinued if a new diagnosis of interstitial lung disease is confirmed.

### Example 8: Liposomal Irinotecan Manufacturing

The liposomal irinotecans can be prepared in a multi-step process. First, lipids are dissolved in heated ethanol. The lipids can include DSPC, cholesterol and MPEG-2000-DSPE combined in a 3:2:0.015 molar ratio. Preferably, the liposomes can encapsulate irinotecan sucrose octasulfate (SOS) encapsulated in a vesicle consisting of DSPC, cholesterol and MPEG-2000-DSPE combined in a 3:2:0.015 molar ratio. The resulting ethanol-lipid solution is dispersed in an aqueous medium containing substituted amine and polyanion under conditions effective to form a properly sized (e.g. 80-120 nm) essentially unilamellar liposome containing the substituted amine (in the ammonium form) and polyanion encapsulated within a vesicle formed from the dissolved lipids. The dispersing can be performed, e.g., by mixing the ethanolic lipid solution with the aqueous solution containing a substituted amine and polyanion at the temperature above the lipid transition temperature, e.g., 60-70° C., and extruding the resulting hydrated lipid suspension (multilamellar liposomes) under pressure through one or more track-etched, e.g. polycarbonate, membrane filters with defined pore size, e.g. 50 nm, 80 nm, 100 nm, or 200 nm. The substituted amine can be triethylamine (TEA) and the polyanion can be sucrose octasulfate (SOS) combined in a stoichiometric ratio (e.g., TEA8SOS) at a concentration of about 0.4-0.5N. All or substantially all non-entrapped TEA or SOS is then removed (e.g., by gel-filtration, dialysis or ultrafiltration) prior to contacting the liposome with irinotecan under conditions effective to allow the irinotecan to enter the liposome in exchange with TEA leaving the liposome. The conditions can include one or more conditions selected from the group consisting of: addition of the osmotic agent (e.g., 5% dextrose) to the liposome external medium to balance the osmolality of the entrapped TEA-SOS solution and/or prevent osmotic rupture of the liposomes during the loading, adjustment and/or selection of the pH (e.g. to 6.5) to reduce the drug and/or lipid degradation during the loading step, and increase of the temperature above the transition temperature of the liposome lipids (e.g., to 60-70° C.) to accelerate the transmembrane exchange of TEA and irinotecan. The loading of irinotecan by exchange with TEA across the liposome preferably continues until all or substantially all of the TEA is removed from the liposome, thereby exhausting its concentration gradient across the liposome. Preferably, the irinotecan liposome loading process continues until the gram-equivalent ratio of irinotecan to sucrooctasulfate is at least 0.9, at least 0.95, 0.98, 0.99 or 1.0 (or ranges from about 0.9-1.0, 0.95-1.0, 0.98-1.0 or 0.99-1.0). Preferably, the irinotecan liposome loading process continues until the TEA is at least 90%, at least 95%, at least 98%, at least 99% or more of the TEA is removed from the liposome interior. The irinotecan can form irinotecan sucrosofate within the liposome, such as irinotecan and sucrose octasulfate in a molar ratio of about 8:1. Next, any remaining extra-liposomal irinotecan and TEA is removed to obtain the irinotecan liposome using, e.g., gel (size exclusion) chromatography, dialysis, ion exchange, or ultrafiltration methods. The liposome external medium is replaced with injectable, pharmacologically acceptable fluid, e.g., buffered isotonic saline. Finally, the liposome composition is sterilized, e.g., by 0.2-micron filtration, dispensed into dose vials, labeled and stored, e.g., upon refrigeration at 2-8° C., until use. The liposome external medium can be replaced with pharmacologically acceptable fluid at the same time as the remaining extra-liposomal irinotecan and TEA is removed. The extra-liposomal pH of the composition can be adjusted or otherwise selected to provide a desired storage stability property (e.g., to reduce formation of the lyso-PC within the liposome during storage at 4° C. over 180 days), for example by preparing the composition at a pH of about 6.5-8.0, or any suitable pH value there between (including, e.g., 7.0-8.0, and 7.25). Irinotecan liposomes with the extra-liposomal pH values, irinotecan free base concentration (mg/mL) and various concentrations of sucrose octasulfate can be prepared as provided in more detail as described herein.

DSPC, cholesterol (Chol), and PEG-DSPE were weighed out in amounts that corresponded to a 3:2:0.015 molar ratio, respectively (e.g., 1264 mg/412.5 mg/22.44 mg). The lipids were dissolved in chloroform/methanol (4/1 v/v), mixed thoroughly, and divided into 4 aliquots (A-D). Each sample was evaporated to dryness using a rotary evaporator at 60° C. Residual chloroform was removed from the lipids by placing under vacuum (180 μtorr) at room temperature for 12 h. The dried lipids were dissolved in ethanol at 60° C., and pre-warmed TEA8SOS of appropriate concentration was added so that the final alcohol content was 10% (v/v). The lipid concentration was 75 mM. The lipid dispersion was extruded at about 65° C. through 2 stacked 0.1 μm polycarbonate membranes (Nucleopore) 10 times using Lipex thermobarrel extruder (Northern Lipids, Canada), to produce liposomes with a typical average diameter of 95-115 nm (determined by quasielastic light scattering). The pH of the extruded liposomes was adjusted with 1 N NaOH to pH 6.5 as necessary. The liposomes were purified by a combination of ion-exchange chromatography and size-exclusion chromatography. First, Dowex™ IRA 910 resin was treated with 1 N NaOH, followed by 3 washes with deionized water and then followed by 3 washes of 3 N HCl, and then multiple washes with water. The liposomes were passed through the prepared resin, and the conductivity of the eluted fractions was measured by using a flow-cell conductivity meter (Pharmacia, Upsalla, Sweden). The fractions were deemed acceptable for further purification if the conductivity was less than 15 μS/cm. The liposome eluate was then applied to a Sephadex G-75 (Pharmacia) column equilibrated with deionized water, and the collected liposome fraction was measured for conductivity (typically less than 1 μS/cm). Cross-membrane isotonicity was achieved by addition of 40% dextrose solution to a final concentration of 5% (w/w) and the buffer (Hepes) added from a stock solution (0.5 M, pH 6.5) to a final concentration of 10 mM.

A stock solution of irinotecan was prepared by dissolving irinotecan.HCl trihydrate powder in deionized water to 15 mg/mL of anhydrous irinotecan-HCl, taking into account water content and levels of impurities obtained from the certificate of analysis of each batch. Drug loading was initiated by adding irinotecan at 500 g/mol liposome phospholipid and heating to 60±0.1° C. for 30 min in a hot water bath. The solutions were rapidly cooled upon removal from the water bath by immersing in ice cold water. Extraliposomal drug was removed by size exclusion chromatography, using Sephadex G75 columns equilibrated and eluted with Hepes buffered saline (10 mM Hepes, 145 mM NaCl, pH 6.5). The samples were analyzed for irinotecan by HPLC and phosphate by the method of Bartlett (see Phosphate Determination). For storage, the samples were divided into 4 mL aliquots and the pH was adjusted as indicated in the Results using 1 N HCl or 1 N NaOH, sterile filtered under aseptic conditions, and filled into sterile clear glass vials that were sealed under argon with a Teflon® lined threaded cap and placed in a thermostatically controlled refrigerator at 4° C. At defined time points, an aliquot was removed from each sample and tested for appearance, size, drug/lipid ratio, and drug and lipid chemical stability. The liposome size was determined in the diluted samples by dynamic light scattering using Coulter Nano-Sizer at 90 degree angle, and presented as Mean±Standard deviation (nm) obtained by the method of cumulants.

### Example 9: ONIVYDE (MM-398) Liposomal Irinotecan

One preferred example of a storage stable liposomal irinotecan described herein is the product that will be marketed as ONIVYDE (irinotecan liposome injection). ONIVYDE is a topoisomerase inhibitor, formulated with irinotecan hydrochloride trihydrate into a liposomal dispersion, for intravenous use. ONIVYDE indicated for the treatment of metastatic adenocarcinoma of the pancreas after disease progression following gemcitabine-based therapy.

ONIVYDE is a storage stabilized liposome having a pH of about 7.25. The ONIVYDE product contains irinotecan sucrosofate encapsulated in a liposome, obtained from an irinotecan hydrochloride trihydrate starting material. The chemical name of irinotecan is (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate. The dosage of ONIVYDE can be calculated based on the equivalent amount of irinotecan trihydrate hydrochloride starting material used to prepare the irinotecan liposomes, or based on the amount of irinotecan in the liposome. There are about 866 mg of irinotecan per gram of irinotecan trihydrate hydrochloride. For example, an ONIVYDE dose of 80 mg based on the amount of irinotecan hydrochloride trihydrate starting material actually contains about 0.866×(80 mg) of irinotecan free base in the final product (i.e., a dose of 80 mg/m² of ONIVYDE based on the weight of irinotecan hydrochloride starting material is equivalent to about 70 mg/m² of irinotecan free base in the final product). ONIVYDE is a sterile, white to slightly yellow opaque isotonic liposomal dispersion. Each 10 mL single-dose vial contains 43 mg irinotecan free base at a concentration of 4.3 mg/mL. The liposome is a unilamellar lipid bilayer vesicle, approximately 110 nm in diameter, which encapsulates an aqueous space containing irinotecan in a gelated or precipitated state as the sucrose octasulfate salt. The vesicle is composed of 1,2-distearoyl-sn-glycero-3-phosphocholine (DSPC) 6.81 mg/mL, cholesterol 2.22 mg/mL, and methoxy-terminated polyethylene glycol (MW 2000)-distearoylphosphatidyl ethanolamine (MPEG-2000-DSPE) 0.12 mg/mL. Each mL also contains 2-[4-(2-hydroxyethyl) piperazin-1-yl]ethanesulfonic acid (HEPES) as a buffer 4.05 mg/mL and sodium chloride as an isotonicity reagent 8.42 mg/mL. Each vial of ONIVYDE contains 43 mg/10 mL irinotecan free base as a white to slightly yellow, opaque, liposomal dispersion in a single-dose vial.

In one example, an ONIVYDE unit dosage form is a pharmaceutical composition comprising an amount of irinotecan encapsulated liposomes that provide a total amount of about 90 mg/m² of irinotecan free base, or an amount of irinotecan equivalent to 100 mg/m² irinotecan hydrochloride trihydrate. The unit dosage form can be an intravenous formulation obtained by diluting a unit dosage form (e.g., a vial) at a concentration of about 4.3 mg irinotecan free base/mL injectable fluid into a total volume of about 500 mL. ONIVYDE is prepared for administering by diluting the isotonic liposomal dispersion from the vial as follows: withdraw the calculated volume of ONIVYDE from the vial. ONIVYDE is diluted into 500 mL 5% Dextrose Injection, USP or 0.9% Sodium Chloride Injection, USP and mix diluted solution by gentle inversion; protect diluted solution from light and administer diluted solution within 4 hours of preparation when stored at room temperature or within 24 hours of preparation when stored under refrigerated conditions [2° C. to 8° C. (36° F. to 46° F.)].

### Example 10

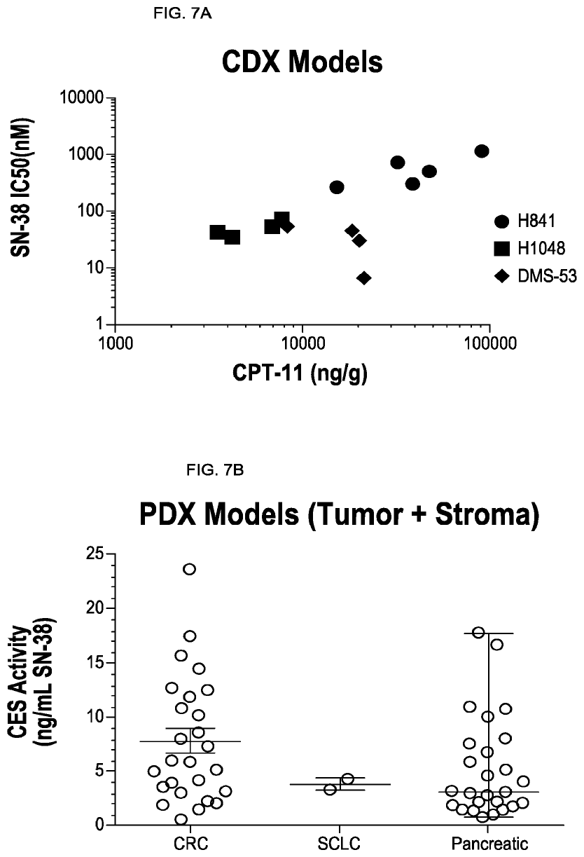

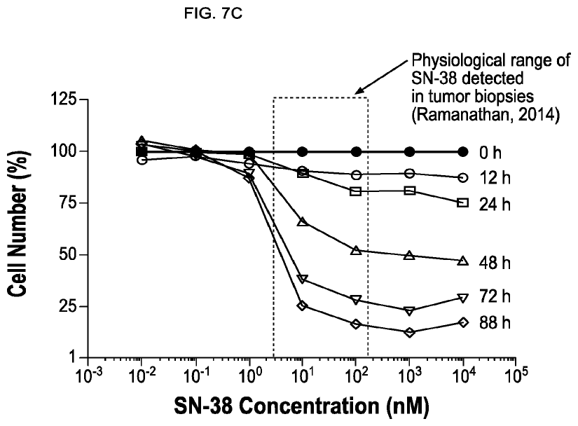

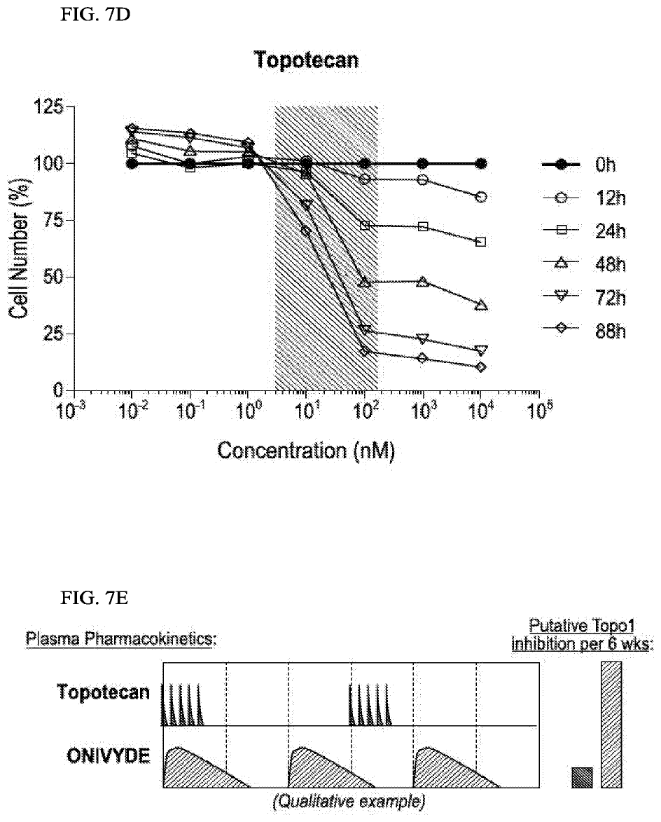

The ability of Nal-IRI to deliver irinotecan and SN-38 to tumors was evaluated in SCLC cell-line derived xenograft (CDX) models (NCI-H1048, DMS-114, H841) in comparison to patient-derived xenograft (PDX) models (CRC, SCLC and pancreatic). Irinotecan liposome injection was administered intravenously to mice bearing xenograft tumors. At 24 hours post administration, mice were sacrificed and tumors were harvested. Irinotecan and SN-38 in tumors were measured by high performance liquid chromatography (HPLC). Data were normalized to injected dose per tumor weight. FIG. 7A shows the increased tumor SN-38 levels were associated with increased tumor deposition, as assessed by tumor CPT-11 at 24 hours post administration in SCLC mouse xenograft models (H841, H1048 and DMS-53). FIG. 7B shows the carboxylesterase (CES) activity in CRC, SCLC, and pancreatic PDX tumors showing that SCLC PDX tumors have CES activity comparable to other indications in which irinotecan is active. Treatment with SN-38 decreased cell viability by >90% in SCLC cell lines (DMS114, NCI-H1048). As shown in FIG. 7C (for NCI-H1048 cells) effective cell growth inhibition was observed between 1-10 nM, with increased cell killing with increased time of exposure over a time-course of up to 88 hours. The concentration range of SN-38 in which cell killing begins to occur coincides with the amount of SN-38 measured from tumor biopsies taken from patients with various solid tumors 72 hours after administration of irinotecan liposome injection (range: 3-163 nM; Ramanathan et al., Eur. J. Cancer, 2014 November; 50:87) is overlaid on the time-dependent SN-38 growth inhibition curves (shown in the area within the dashed lines). Similar effects were seen in DMS-114 cells. Cell growth inhibition kinetics of SN-38 in the cell lines was determined using IncuCyte® ZOOM System. FIG. 7D is a graph showing cell sensitivity; cytotoxicity of Topo1 inhibitors increases with exposure. FIG. 7E is a chart showing that topotecan administration is severely limited by toxicity, thus limiting sustained inhibition of topo1 in comparison to Onivyde mediated prolonged SN-38 exposure.

### Example 11. Preclinical Support for Evaluation of Irinotecan Liposome Injection (Nal-IRI, MM-398) in Patients with Small Cell Lung Cancer

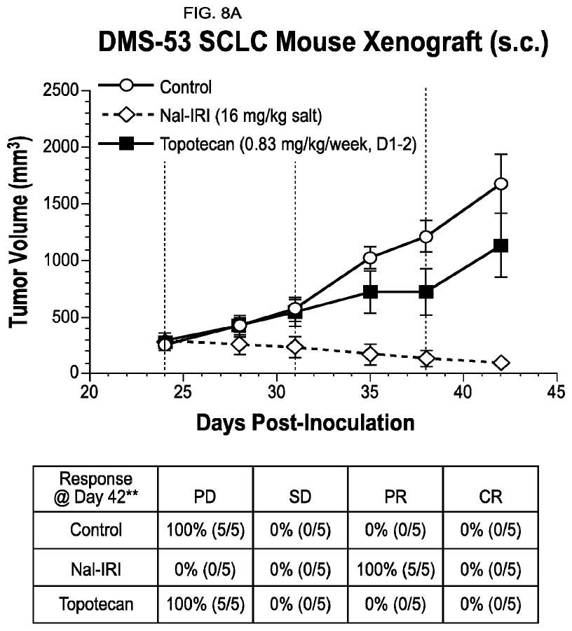

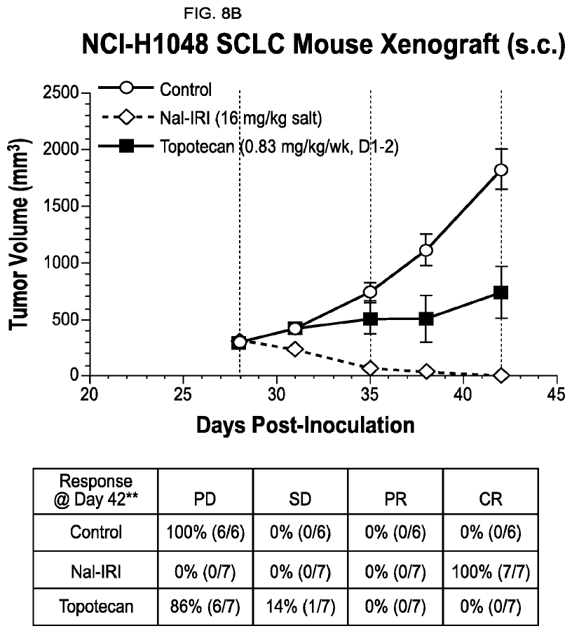

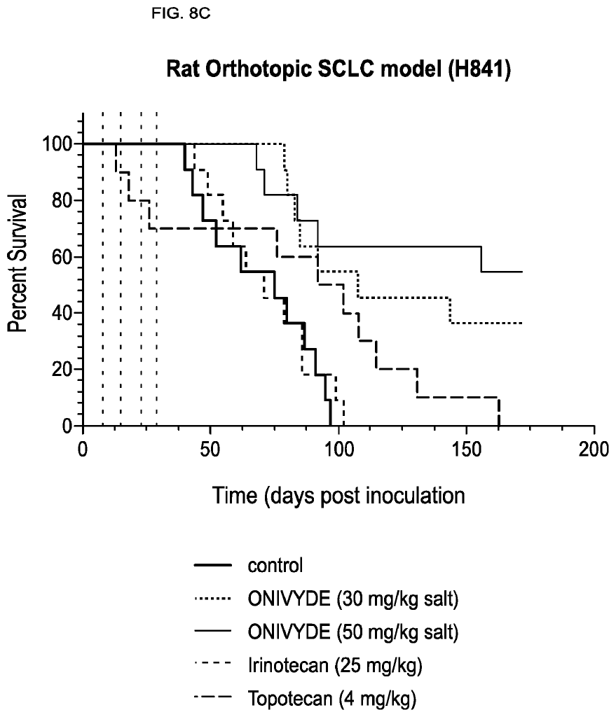

Anti-tumor activity of nal-IRI as a monotherapy was evaluated in DMS-53 and NCI-H1048 xenograft models. Cells were implanted subcutaneously into right flanks of NOD-SCID mice; treatments were initiated when tumors had reached approximately 280 mm³. Nal-IRI was dosed at 16 mg/kg salt, q1w, which is equivalent to a proposed clinical dose of 90 mg/m² free base, q2w. Topotecan was dosed at 0.83 mg/kg/week, Day 1-2 every 7 days, which approximates a clinical dose intensity of 1.5 mg/m² (Day 1-5 every 21 days). Tumor metabolite levels of nal-IRI and non-liposomal irinotecan were measured at 24 hours post-injection, using previously established high performance liquid chromatography methods. The results for the monotherapy treatment in DMS-53 are shown in FIG. 8A and the results in NCI-H1048 are shown in FIG. 8B. In FIGS. 8A and 8B the vertical dotted lines indicate days of dosing and the response rates are determined based on tumor volume change from base line: CR: change in tumor volume (TV)<−95%; PR: −95%≤change in TV <−30%; SD: −30%≤change in TV<30%; PD: change in TV≥30%. Nal-IRI displayed significantly greater anti-tumor activity than topotecan based on tumor growth kinetics and overall survival. Furthermore, 7 out of 7 mice in NCI-H1048 model treated with nal-IRI experienced complete tumor regressions after 4 cycles of treatment and maintained for at least 50 days after last dose, compared to 0 out of 7 mice treated with topotecan.

Carboxylesterase activity and sensitivity to SN-38 in SCLC models were comparable to that in indications where nal-IRI or irinotecan HCl has proven to be efficacious clinically (e.g. pancreatic cancer, colorectal cancer). Nal-IRI was found to deliver irinotecan to tumors in SCLC tumors to a similar or greater extent than other tumor types. The tumor irinotecan and SN-38 levels of nal-IRI (16 mg/kg salt) were 12 to 57-fold and 5 to 20-fold higher than nonliposomal irinotecan (30 mg/kg salt), respectively. Nal-IRI demonstrated anti-tumor activity in both xenograft models of SCLC at clinically relevant dose levels, and resulted in complete or partial responses after 4 cycles of treatment, compared to topotecan which have limited tumor growth control.

The anti-tumor activity of MM-398 (Onivyde) in the H841 rat orthotopic xenograft model of SCLC is shown in FIG. 8C which is a graph showing the percent survival of rats treated with control, Onivyde (30 or 50 mg/kg salt), irinotecan (25 mg/kg) or topotecan (4 mg/kg) for days post inoculation. Rats treated with Onivyde at both 30 and 50 mg/kg showed longer survival times that those treated with control, irinotecan or topotecan. MM-398 has anti-tumor activity in multiple SCLC xenograft models. At clinically relevant doses (16 mg/kg/wk MM-398, 0.8 mg/kg/wk topotecan), MM-398 had greater anti-tumor activity and prolonged survival than topotecan.

These studies demonstrated that nal-IRI is more active than topotecan at clinically relevant doses in SCLC preclinical models, and thus support a proposed randomized Phase 3 trial of nal-IRI versus topotecan in patients with SCLC that have progressed on prior platinum-based therapy.

### Example 12

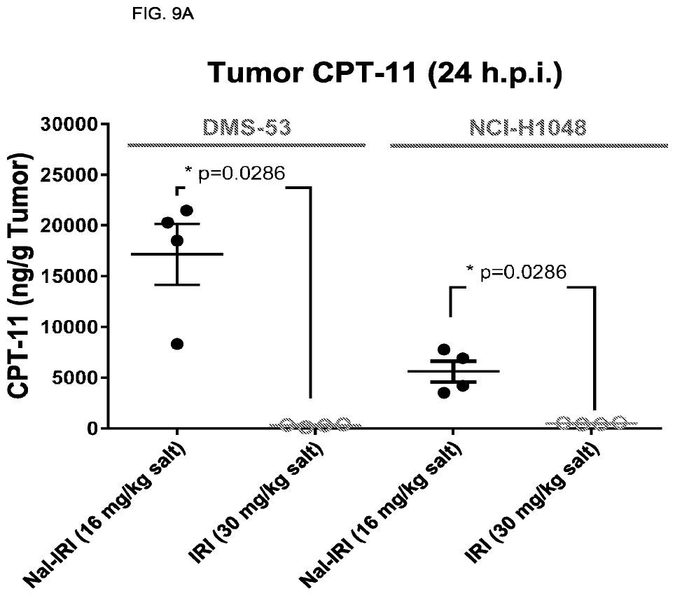

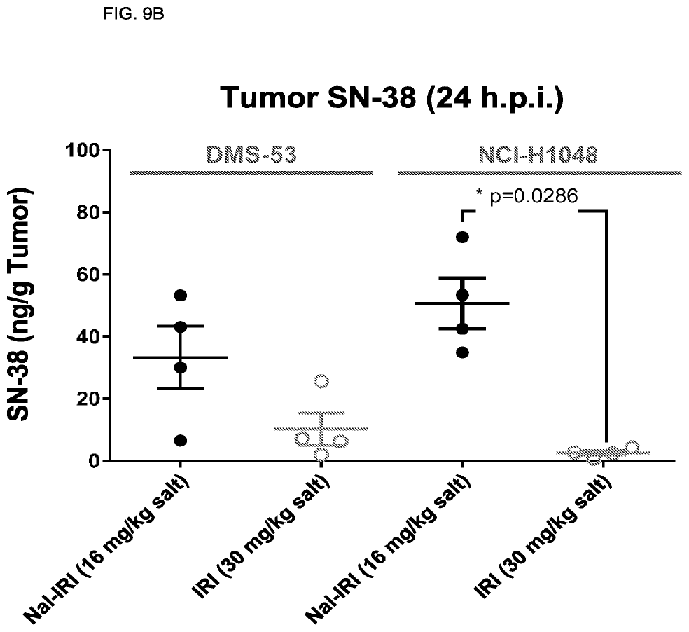

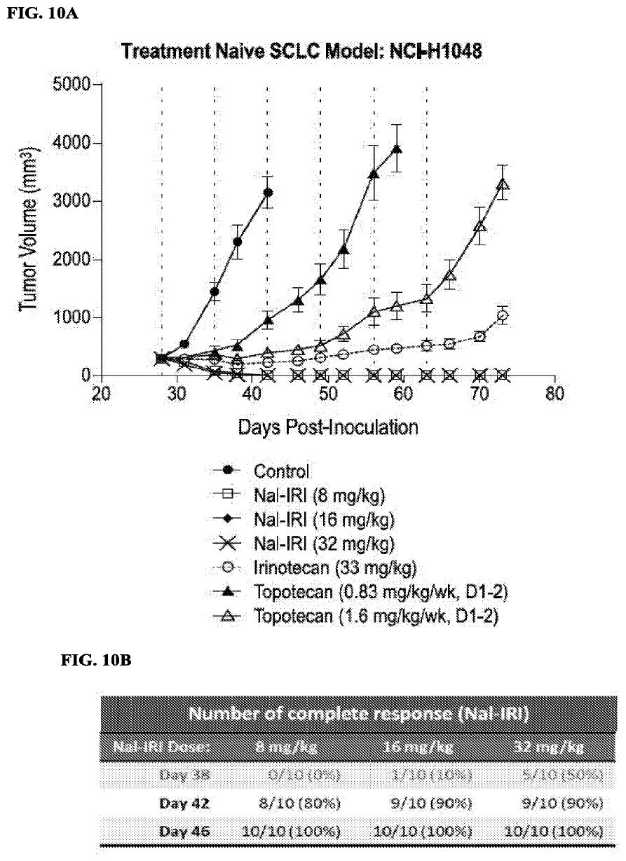

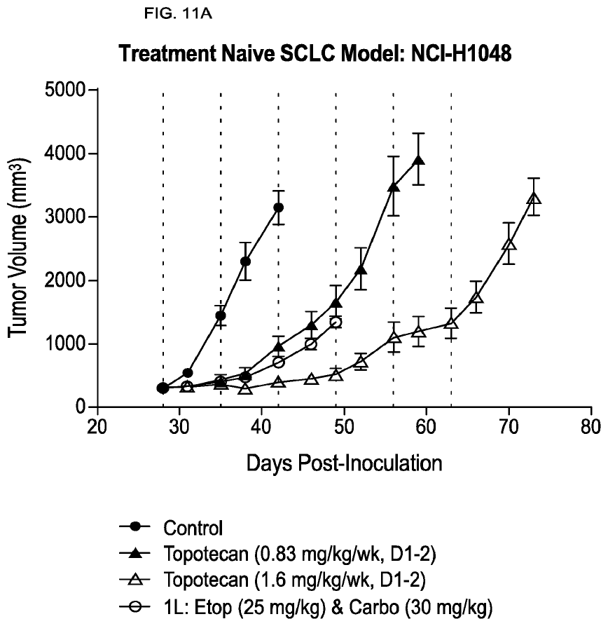

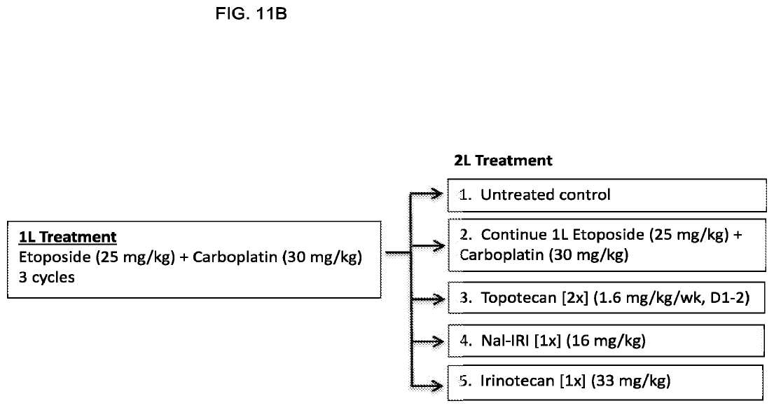

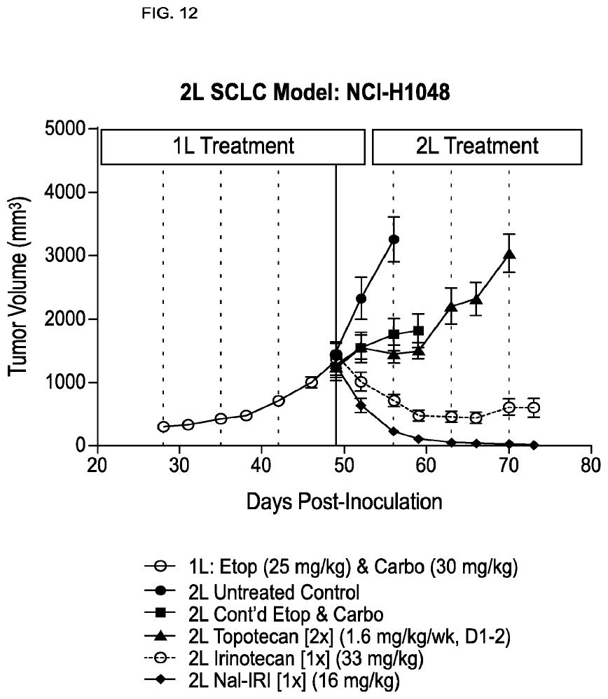

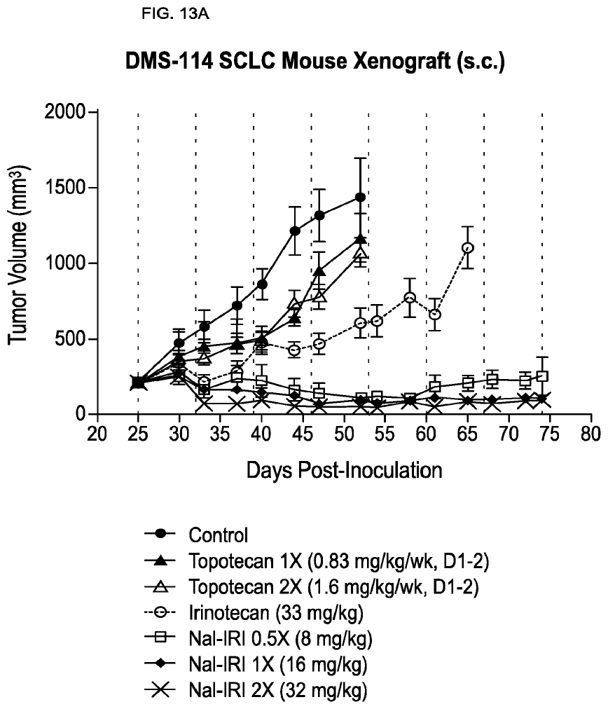

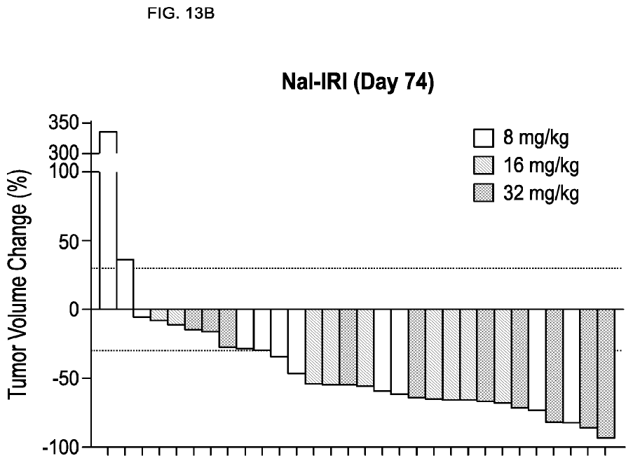

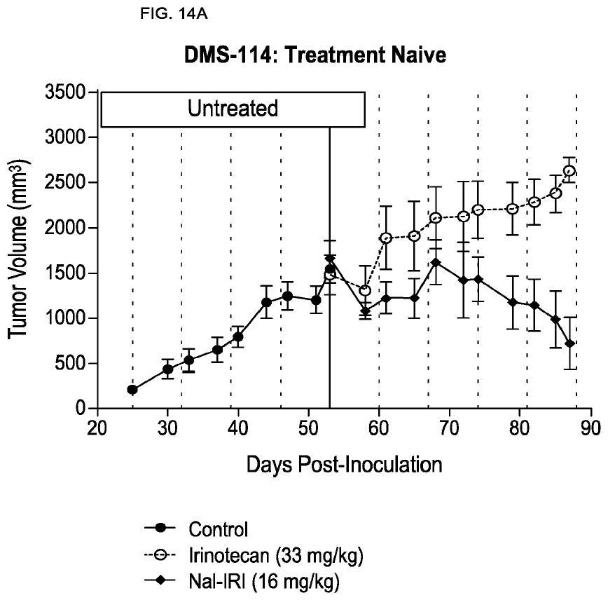

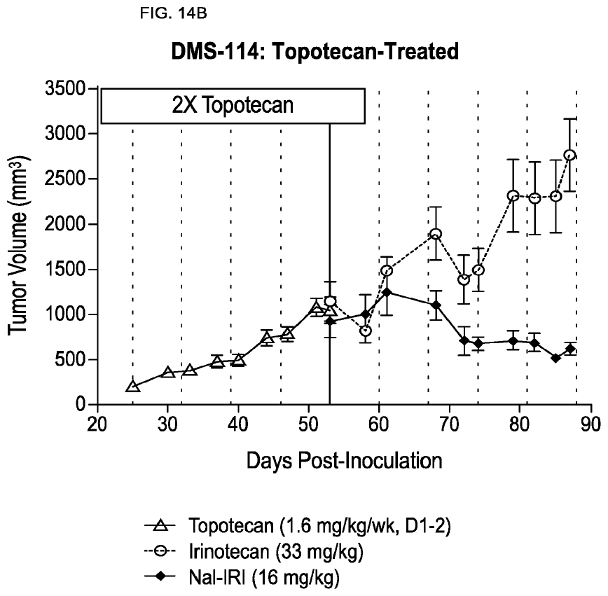

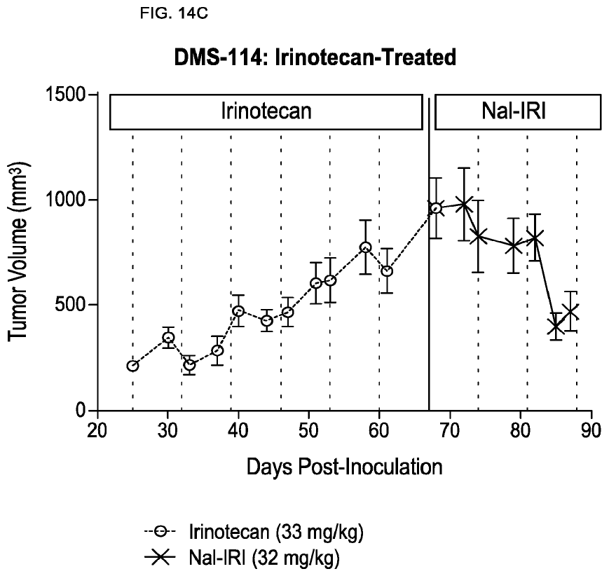

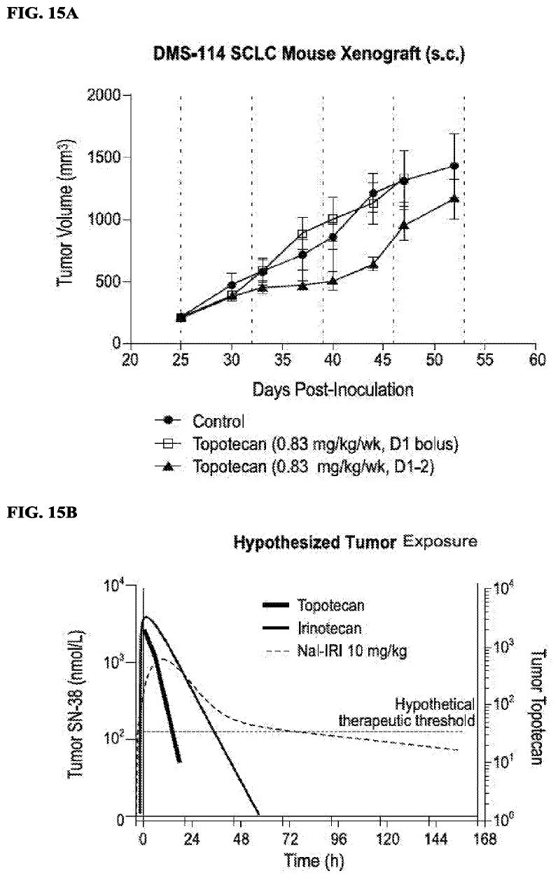

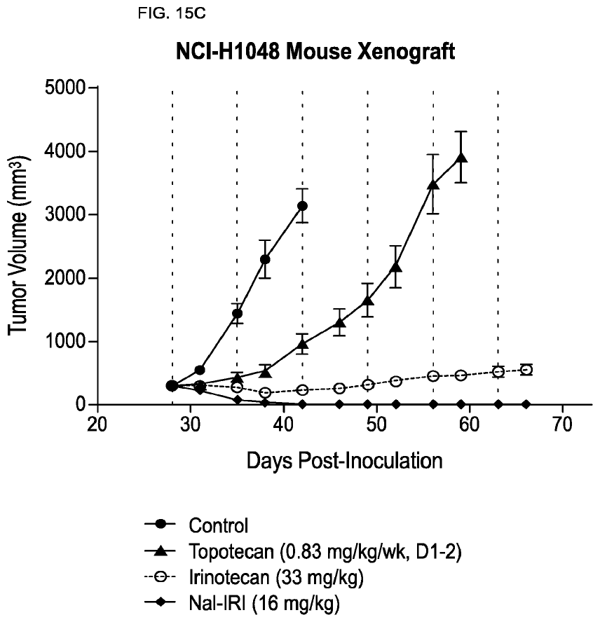

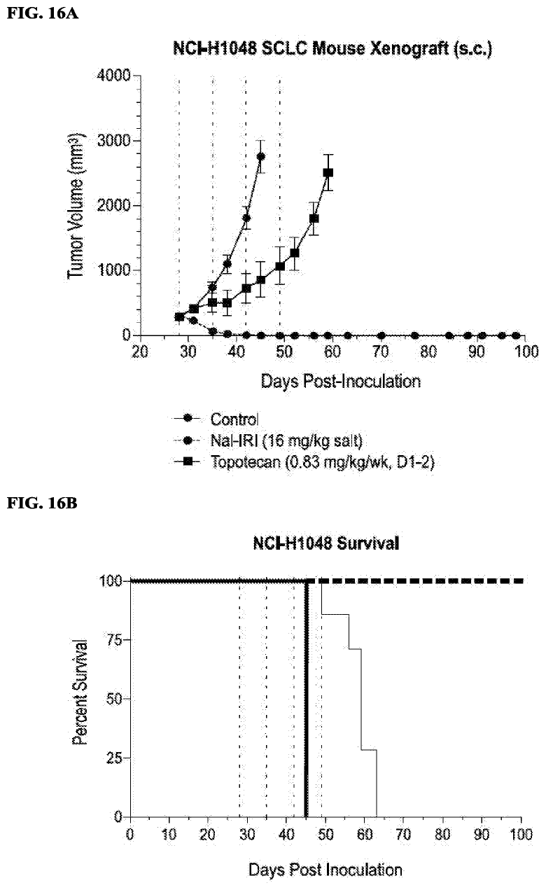

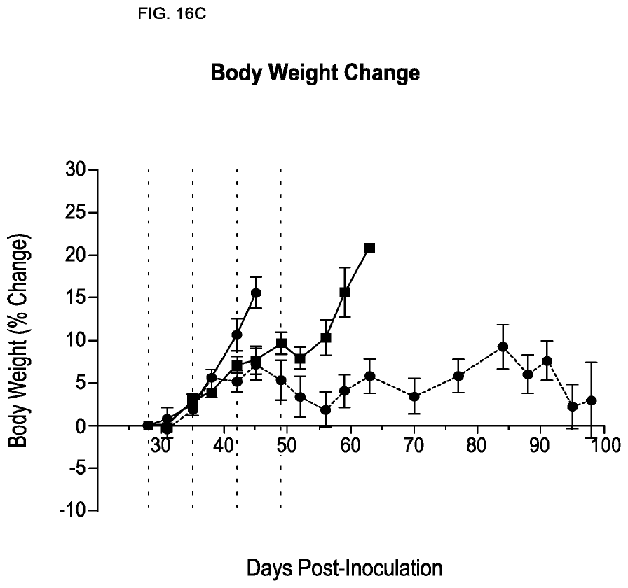

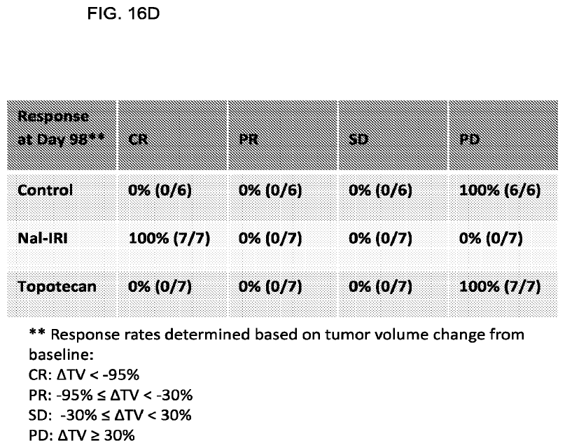

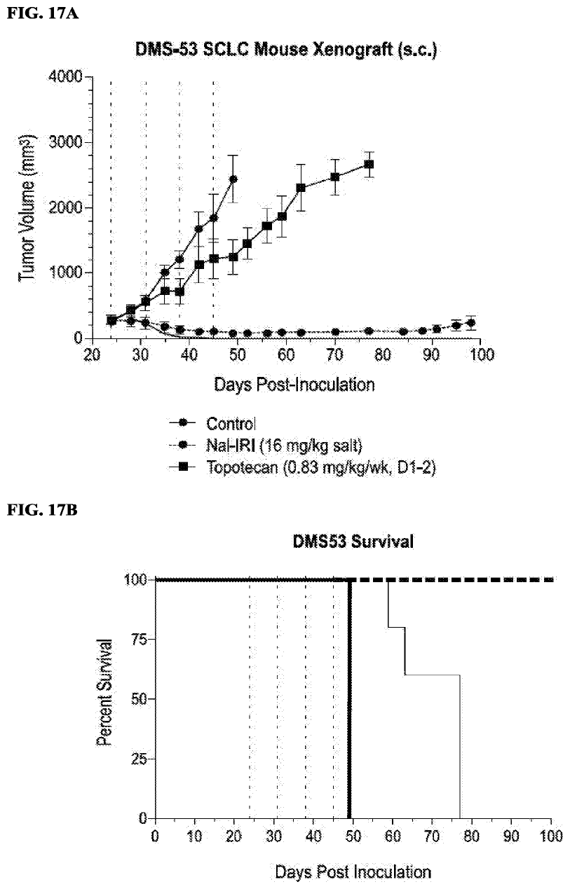

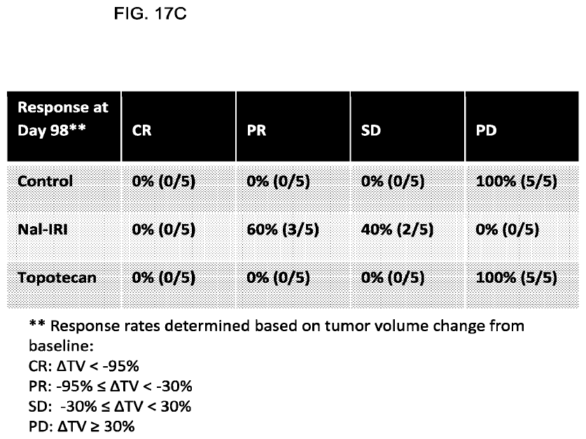

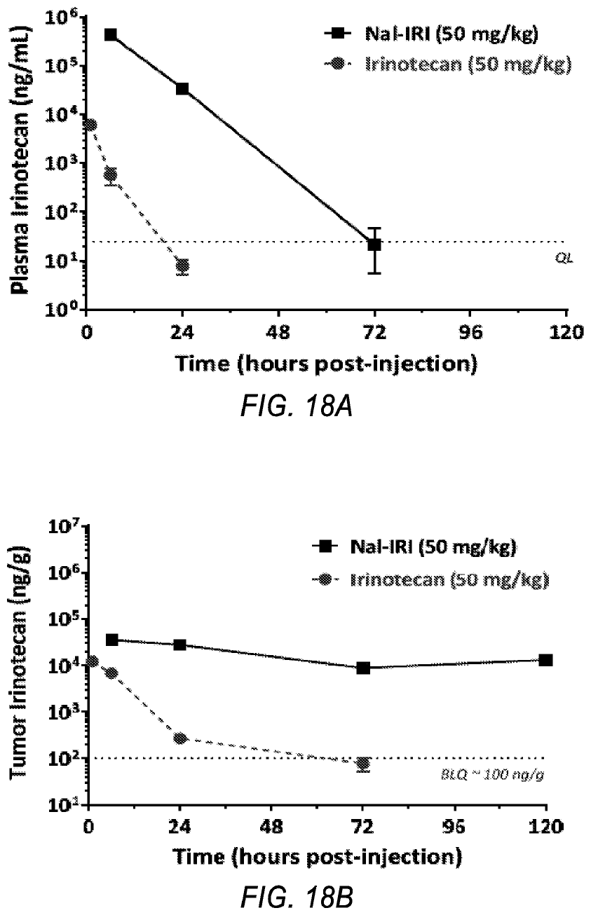

Tumor metabolites levels of nal-IRI were compared to non-liposomal irinotecan in SCLC tumor bearing xenograft models DMS-53 and NCI-H1048 (FIGS. 9A and 9B). Based on body-surface area dosing and scaled to body weight, the clinically-relevant doses of nal-IRI and non-liposomal irinotecan HCl in mice are approximately 16 mg/kg (salt) and 30 mg/kg (salt), respectively. Nal-IRI dosed at 16 mg/kg salt (q1w) is equivalent to a proposed clinical dose of 90 mg/m² free base, q2w. Irinotecan HCl dosed at 30 mg/kg, q1w, approximates a clinical dose intensity of 300 mg/m², q3w, which resulted in similar efficacy as topotecan (current standard of care) in second-line SCLC patients (Zhao M L, Bi Q, Ren H X, Tian Q, Bao M L. Clinical observation of irinotecan or topotecan as second-line chemotherapy on treating 43 patients with small-cell lung cancer. Chin Oncol. 2011; 21:156-158).

Using high performance liquid chromatography methods, tumor levels of CPT-11 (FIG. 9A) and the active metabolites SN-38 (FIG. 9B) were measured at 24 hours post-injection (intravenous via tail vein). In both SCLC models, nal-IRI delivered irinotecan to tumors to a greater extent than non-liposomal irinotecan HCl. The tumor CPT-11 and SN-38 levels of nal-IRI (16 mg/kg salt) were 12 to 57-fold and 5 to 20-fold higher than non-liposomal irinotecan (30 mg/kg salt), respectively. The increased tumor CPT-11 and SN-38 delivered by nal-IRI is attributed to the extended circulation as a result of liposomal encapsulation, as well as local activation of liposomal-irinotecan in the tumors (PMID 25273092: Preclinical activity of nanoliposomal irinotecan is governed by tumor deposition and intratumor prodrug conversion. Kalra AV1, Kim J1, Klinz SG1, Paz N1, Cain J1, Drummond DC1, Nielsen UB1, Fitzgerald J B)

### Example 13: Irinotecan Liposome Injection-Mediated Tumor Delivery of Irinotecan and SN-38 In Vivo

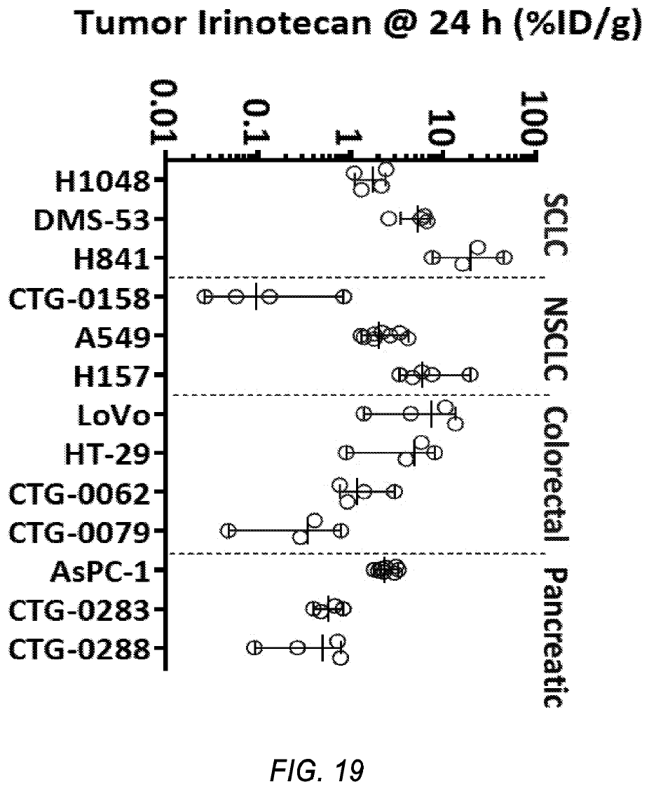

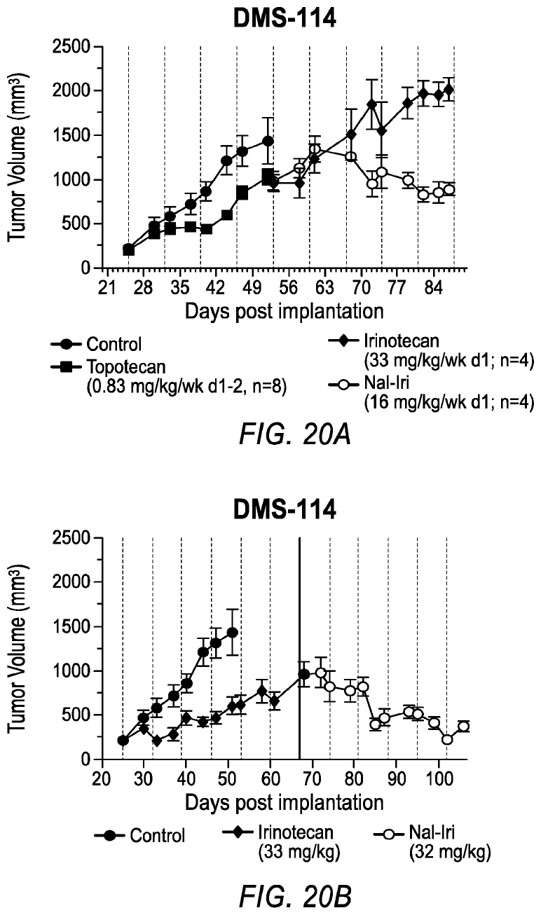

The ability of MM-398 to deliver irinotecan and SN-38 to tumors was evaluated in SCLC cell-line derived xenograft (CDX) models (NCI-H1048, DMS-114, H841) in comparison to CDX and patient-derived xenograft (PDX) models of other tumor types. Irinotecan liposome injection was administered intravenously to mice bearing xenograft tumors. At 24 hours post administration, mice were sacrificed and tumors were harvested. Irinotecan and SN-38 in tumors were measured by high performance liquid chromatography (HPLC). Data were normalized to injected dose per tumor weight. As shown in FIG. 19, tumors derived from SCLC cell lines have similar or higher levels of irinotecan liposome injection deposition, as assessed by irinotecan content, than other tumor types. Furthermore, analysis of SN-38 levels indicates that increased irinotecan delivery was associated with increased levels of SN-38. These findings are consistent with a proposed mechanism of liposome deposition and local conversion of irinotecan to SN-38 within the tumor.

### Example 14: Anti-Tumor Activity of Irinotecan Liposome Injection, Non-Liposomal Irinotecan and Topotecan in a Preclinical Model of Second Line SCLC

Nal-IRI is designed for extended circulation relative to non-liposomal irinotecan and to exploit leaky tumor vasculature for enhanced drug delivery to tumors. Following tumor deposition, nal-IRI is taken up by phagocytic cells followed by irinotecan release and conversion to its active metabolite, SN-38, in the tumors. Sustained inhibition of topoisomerase 1 (TOP1) by extended SN-38 delivery is hypothesized to enable superior anti-tumor activity compared to traditional TOP1 inhibitors. Topotecan, a TOP1 inhibitor, is currently a standard of care for second-line treatment of small cell lung cancer (SCLC).

As described below, mice bearing NCI-H1048 SCLC tumors were treated with a carboplatin plus etoposide, a first line regimen in SCLC. Once the tumors escaped growth control by carboplatin plus etoposide, mice were randomized to either continue treatment with carboplatin plus etoposide or switch to second line treatment with either irinotecan liposome injection, non-liposomal irinotecan or topotecan.

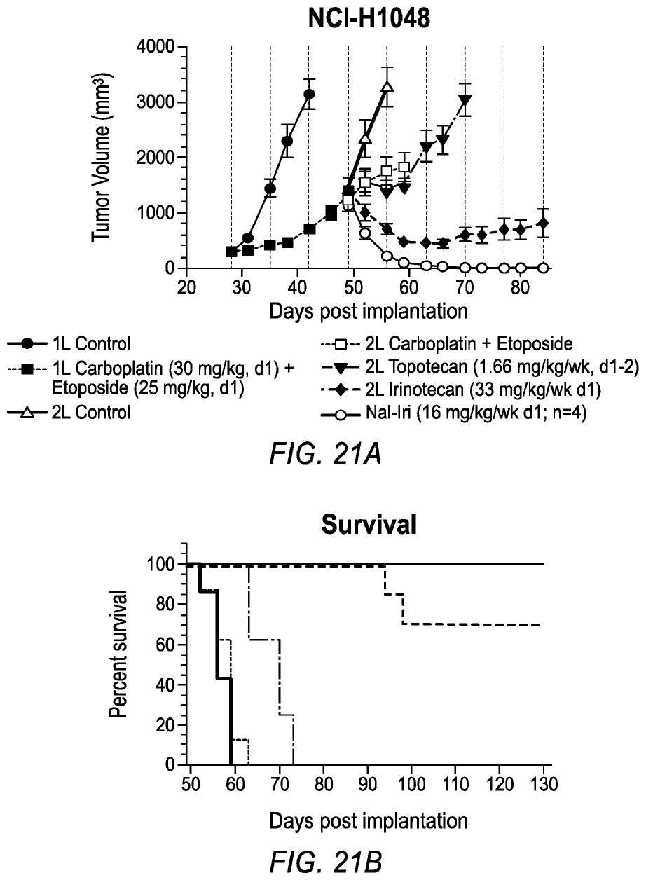

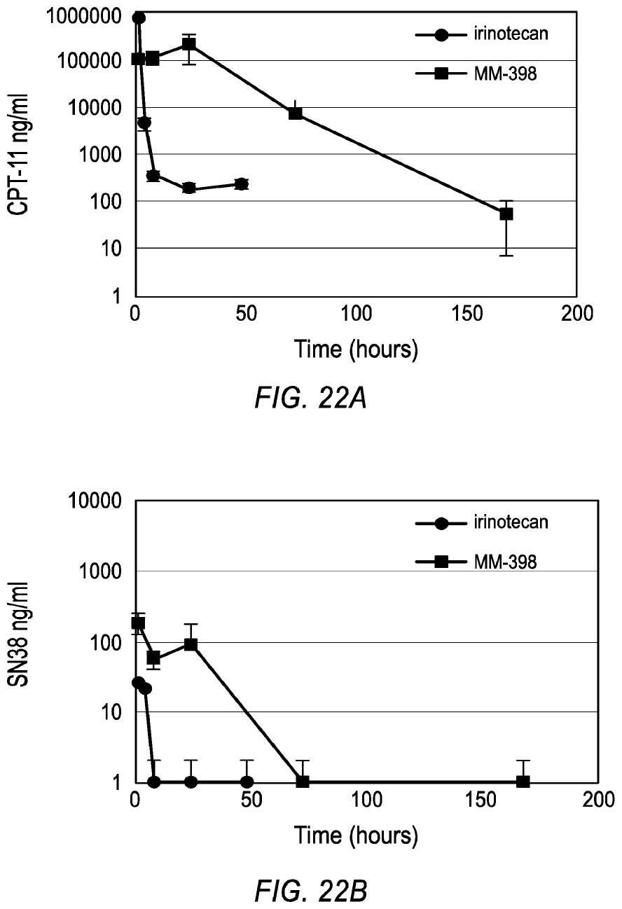

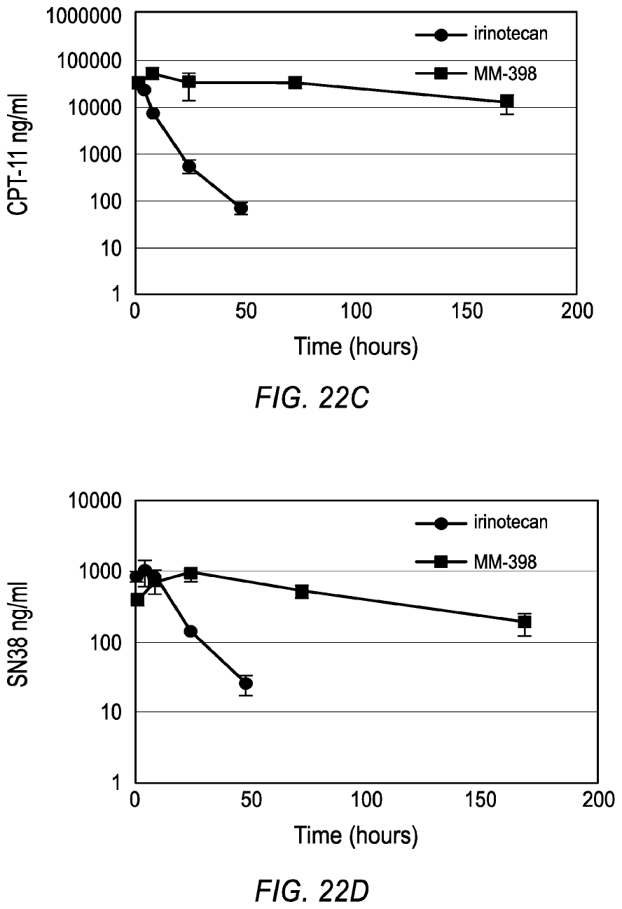

NOD/SCID mice with NCIH1048 SCLC xenograft tumors were treated weekly with the combination of 30 mg/kg carboplatin plus 25 mg/kg etoposide. When tumor reached approximately 1200 mm³, mice were randomized to receive weekly treatment with topotecan (1.66 mg/kg/wk administered IP in equal fractions on days 1 and 2), non-liposomal irinotecan (33 mg/kg/wk administered IV on day 1) irinotecan liposome injection (16 mg/kg/wk administered IV on day 1), continue treatment with carboplatin plus etoposide or vehicle control. Vertical dotted lines indicate start of weekly dosing. Irinotecan liposome injection dose is shown on irinotecan HCl basis. After tumors progressed on first-line treatment with carboplatin plus etoposide, irinotecan liposome injection displayed significant anti-tumor activity compared to topotecan and irinotecan (p=0.0002 on day 70 and p=0.0002 on day 84 for topotecan and irinotecan, respectively). In carboplatin plus etoposide-treated SCLC tumors: Nal-IRI remains active and is trending towards complete response; non-liposomal irinotecan treatment is active but after 3rd cycle some tumors are trending regrowth; Topotecan (at 2× clinically relevant dose) seems to be active after 1-2 cycles but progress quickly after 3rd dose; carboplatin plus etoposide is not tolerable by the 5th cycle. As shown in FIG. 21A, irinotecan liposome injection had anti-tumor activity in the second line setting and, furthermore, had significantly greater anti-tumor activity than both non-liposomal irinotecan and topotecan. FIG. 21B is a survival graph for mice on each of the treatments.

### Example 15: Irinotecan Liposome Injection has Improved Anti-Tumor Activity as Compared to Non-Liposomal Irinotecan HCl and Topotecan In Vivo

The activity of irinotecan liposome injection, non-liposomal irinotecan and topotecan were directly compared at clinically relevant doses in two CDX models (DMS-114 and NCI-H1048) and the activity of irinotecan liposome injection and topotecan in one CDX model (DMS-53). Clinically relevant doses were calculated by using standard surface area to weight ratios conversion per NCI guidelines.

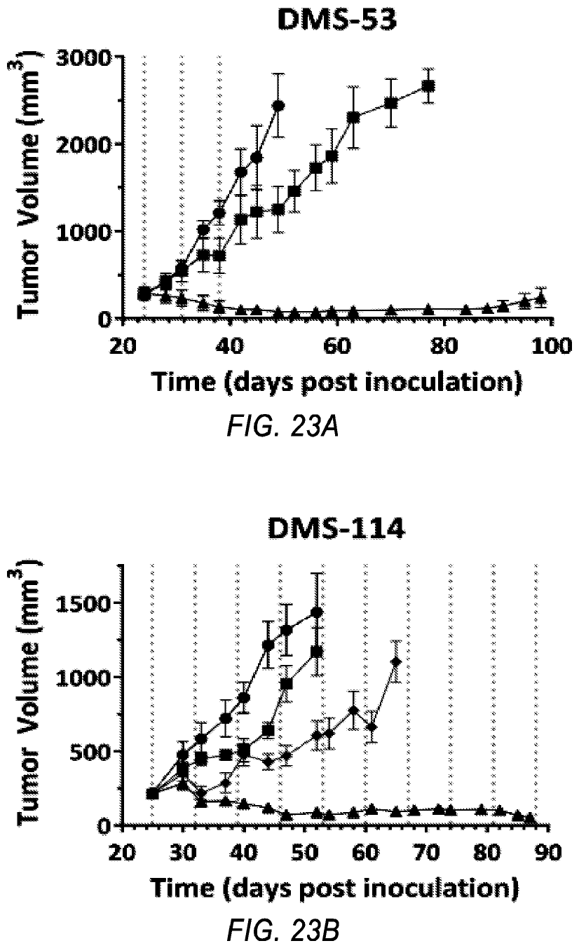

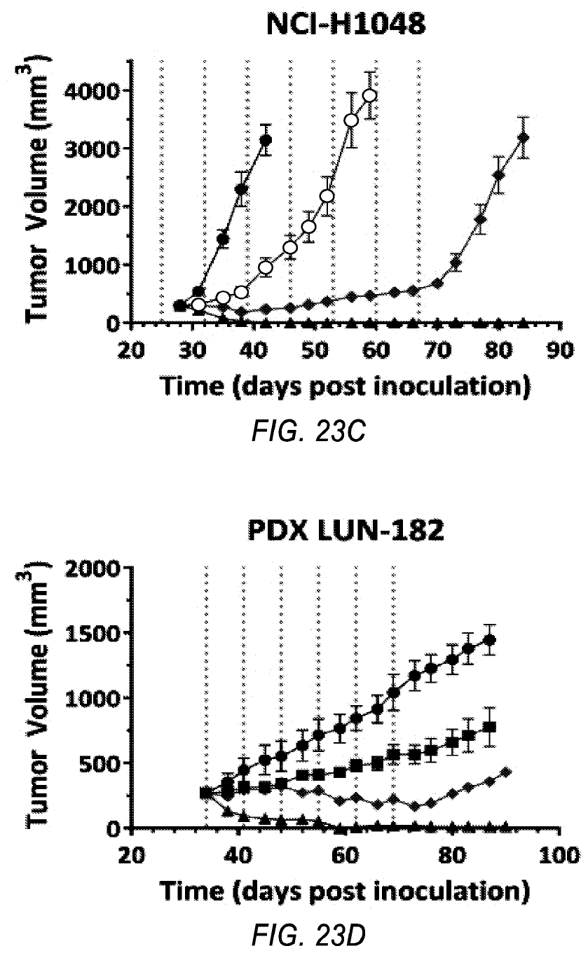

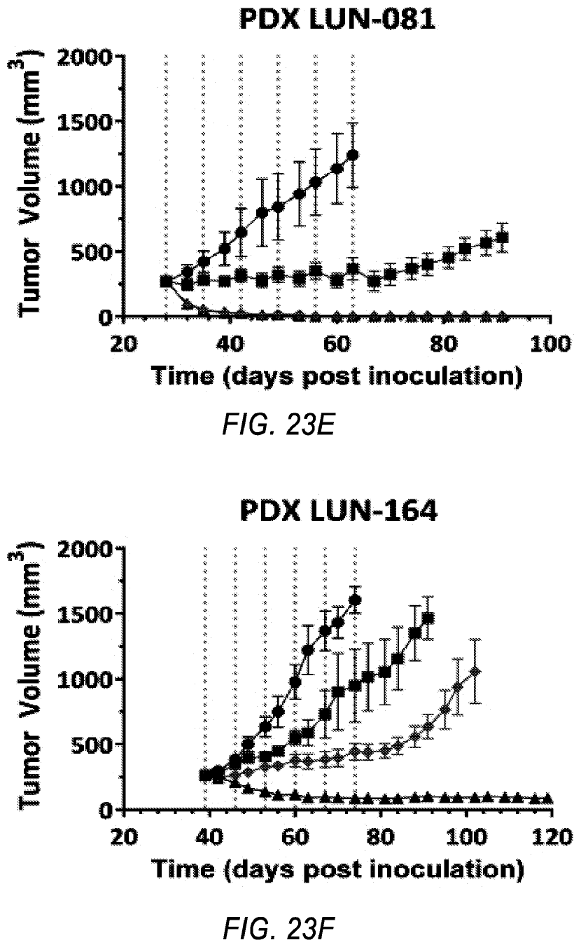

FIG. 23 presents tumor growth kinetics of mice bearing SCLC xenograft tumors that were treated weekly with irinotecan liposome injection, topotecan and non-liposomal irinotecan (two of the three). In the DMS-114 and NCI-H1048 models, irinotecan liposome injection displayed significantly greater anti-tumor activity than both non-liposomal irinotecan and topotecan. In the DMS-53 model, irinotecan liposome injection displayed significantly greater anti-tumor activity than did topotecan. Furthermore, 10 out of 10 mice treated in NCI-H1048 model treated with irinotecan liposome injection experienced complete regressions of their tumors as compared to 0 out of 10 mice treated with topotecan.

FIG. 23 shows the data obtained from NOD/SCID mice with subcutaneous (FIG. 23A) DMS-53, (FIG. 23B) DMS-114 or (FIG. 23C) NCI-H1048. SCLC xenograft tumors were treated with IV nal-IRI (16 mg/kg; triangles), IV irinotecan (33 mg/kg; diamonds), IP topotecan (0.83 mg/kg/wk days 1-2; squares) or vehicle control (circles). For DMS-114 and NCI-H1048 all groups have n=10; for DMS-53 n=4, 5 and 5 for control, topotecan and nal-IRI, respectively. Vertical dotted lines indicate start of weekly dosing and error bars indicate standard error of the mean. Irinotecan liposome injection dose is shown on irinotecan HCl basis. Following treatment, irinotecan liposome injection displayed significant anti-tumor activity compared to topotecan (p<0.0001 for DMS-114 on day 52 and p<0.0001 for NCI-H1048 on day 59; non-parametric t-test) and irinotecan (p<0.0001 for DMS-114 on day 65 and p<0.0001 for NCI-H1048 on day 84; non-parametric t-test).

In addition to the CDX models PDX models were also examined using subcutaneous patient-derived xenografts. Balb/c nude mice bearing subcutaneous patient-derived xenografts (FIG. 23D) LUN-182, (FIG. 23E) LUN-081 and (FIG. 24F) LUN-164 were treated with IV nal-IRI (16 mg/kg; triangles), IV irinotecan (33 mg/kg; diamonds), IP topotecan (0.83 mg/kg/wk days 1-2; squares) or vehicle control (circles). For all PDX models n=5 for all groups. Vertical dotted lines indicate start of weekly dosing and error bars indicate standard error of the mean.

1.-20. (canceled)

21. A method of treating a human patient who is homozygous for the UGT1A1*28 allele and is diagnosed with small cell lung cancer (SCLC) following disease progression on or after first-line platinum-based therapy for the SCLC, the method comprising administering to the human patient an antineoplastic therapy once every two weeks, the antineoplastic therapy consisting of liposomal irinotecan in a dose providing the equivalent of 50 mg/m² irinotecan free base in combination with a Chk1 directed therapeutic.

22. The method of claim 21, wherein the Chk1 directed therapeutic is prexasertib.

23. A method of treating a human patient who is homozygous for the UGT1A1*28 allele and is diagnosed with small cell lung cancer (SCLC) following disease progression on or after first-line platinum-based therapy for the SCLC, the method comprising administering to the human patient an antineoplastic therapy once every two weeks, the antineoplastic therapy consisting of liposomal irinotecan in a dose providing the equivalent of 50 mg/m² irinotecan free base in combination with a Topo-2 directed therapeutic.

24. The method of claim 23, wherein the Topo-2 directed therapeutic is aldozurubicin.

25. A method of treating a human patient who is homozygous for the UGT1A1*28 allele and is diagnosed with small cell lung cancer (SCLC) following disease progression on or after first-line platinum-based therapy for the SCLC, the method comprising administering to the human patient an antineoplastic therapy once every two weeks, the antineoplastic therapy consisting of liposomal irinotecan in a dose providing the equivalent of 50 mg/m² irinotecan free base in combination with a Notch ADC compound.

26. The method of claim 25, wherein the Notch ADC compound is Rova-T.

27. The method of claim 21, wherein the platinum-based therapy comprises the prior, discontinued administration of cisplatin or carboplatin to treat the human patient diagnosed with SCLC.

28. The method of claim 22, wherein the platinum-based therapy comprises the prior, discontinued administration of cisplatin or carboplatin to treat the human patient diagnosed with SCLC.

29. The method of claim 23, wherein the platinum-based therapy comprises the prior, discontinued administration of cisplatin or carboplatin to treat the human patient diagnosed with SCLC.

30. The method of claim 24, wherein the platinum-based therapy comprises the prior, discontinued administration of cisplatin or carboplatin to treat the human patient diagnosed with SCLC.

31. The method of claim 25, wherein the platinum-based therapy comprises the prior, discontinued administration of cisplatin or carboplatin to treat the human patient diagnosed with SCLC.

32. The method of claim 26, wherein the platinum-based therapy comprises the prior, discontinued administration of cisplatin or carboplatin to treat the human patient diagnosed with SCLC.
